(19) United States
(12) Patent Application Publication
(10) Pub. No.: US 2008/0014211 A1
(43) Pub. Date: Jan. 17, 2008

(54) Methods to elicit, enhance and sustain immune responses against MHC class I-restricted epitopes, for prophylactic and therapeutic purposes

(75) Inventors: Adrian Ion Bot, Valencia, CA (US); Kent Andrew Smith, Ventura, CA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: MANNKIND CORPORATION, Valencia, CA (US)

(21) Appl. No.: 11/879,078

(22) Filed: Jul. 14, 2007

Related U.S. Application Data
(60) Provisional application No. 60/831,256, filed on Jul. 14, 2006.
(60) Provisional application No. 60/863,332, filed on Oct. 27, 2006.

(51) Int. Cl. A61K 39/00

(52) U.S. Cl. 424/185.1

(57) ABSTRACT

Embodiments of the present invention relate to methods and compositions for inducing, entraining, and/or amplifying the immune response to MHC class-I restricted epitopes of carcinoma antigens to generate an effective anti-cancer immune response. The methods and compositions disclosed herein, can be used for prophylactic or therapeutic purposes. Further embodiments provide methods of treating a cell proliferative disease, such as cancer by providing to a subject in need thereof a therapeutic strategy comprising an immunogenic composition in combination with a chemotherapeutic agent.

### CROSS-REFERENCE TO RELATED APPLICATIONS

The present application claims the benefit of the filing date of U.S. Provisional Patent Application Ser. No. 60/831,256, filed on Jun. 14, 2006, and 60/863,332 filed on Oct. 27, 2006, each of which is hereby incorporated by reference in its entirety.

### FIELD OF THE INVENTION

Embodiments of the invention disclosed herein relate to methods and compositions for combination immunotherapeutic and chemotherapeutic regimens for prophylactic or therapeutic uses. Particular embodiments relate to chemotherapeutic agents, immunogenic compositions, their nature and the order, timing, and route of administration by which they are effectively used.

### BACKGROUND

Globally suppressed T-cell function has been described in many patients with cancer to be a major hurdle for the development of clinically efficient cancer immunotherapy. Inhibition of antitumor immune responses has been mainly linked to inhibitory factors present in cancer patients. A major barrier to successful antitumor vaccination is tolerance of high-avidity T cells specific to tumor antigens.

### SUMMARY OF THE INVENTION

One embodiment of the invention includes a method of immunization including the steps of: contacting a tumor in a patient with a chemotherapeutic agent, wherein the chemotherapeutic agent promotes tumoral inflammation and/or interfering with T-regulatory cell function; and inducing a CTL response, wherein the inducing includes the substeps of delivering to the patient a first composition that includes an immunogen, and the immunogen includes or encodes at least part of a first antigen, and further includes an immunopotentiator; and administering a second composition, including an amplifying peptide, directly to a lymphatic system of the patient, wherein the peptide corresponds to an epitope of said first antigen. Preferably, the contacting and inducing steps result in an enhanced effectiveness of treatment beyond the effectiveness of either of the contacting step or the inducing step alone.

In some embodiments of the invention, the first composition and the second composition are the same. Alternatively, the first composition and the second composition are not the same. In some embodiments, the first composition includes, for example, a nucleic acid encoding the antigen or an immunogenic fragment thereof. In some embodiments the first composition includes a nucleic acid capable of expressing the antigen or an immunogenic fragment thereof in a pAPC. In some embodiments the first composition includes, for example an immunogenic polypeptide and an immunopotentiator, or the like. In some embodiments of the invention the immunogenic polypeptide is the amplifying peptide.

In some embodiments of the invention, the immunogenic polypeptide is the first antigen. In some embodiments the immunopotentiator is a cytokine. In some embodiments the immunopotentiator is a toll-like receptor ligand. In some embodiments the second composition further includes an adjuvant. In some embodiments of the invention the second composition is adjuvant-free and immunopotentiator-free. In some embodiments the delivering substep includes administration to more than one site. In some embodiments the delivering substep includes, for example, direct administration to the lymphatic system of the patient. In some embodiments direct administration to the lymphatic system of the patient includes, for example, direct administration to a lymph node or lymph vessel.

Still further embodiments include generating an antigen-specific tolerogenic or regulatory immune response. The methods can include periodically administering a composition, including an adjuvant-free peptide, directly to the lymphatic system of a patient, wherein the peptide corresponds to an epitope of the antigen, and wherein the patient can be epitopically naïve, and administering a chemotherapeutic agent simultaneously, or after delivering the first or second composition. The methods further can include obtaining, detecting and assaying for a tolerogenic or regulatory T cell immune response. The immune response can assist in treating an inflammatory disorder or cancer, for example. The inflammatory disorder can be, for example, from a class II MHC-restricted immune response. The immune response can include production of an immunosuppressive cytokine, for example, IL-5, IL-10, or TGB-β, and the like. The cancer can be a breast cancer, an ovarian cancer, a pancreatic cancer, a prostate cancer, a colon cancer, a bladder cancer, a lung cancer, a liver cancer, a stomach cancer, a testicular cancer, an uterine cancer, a brain cancer, a lymphatic cancer, a skin cancer, a bone cancer, a kidney cancer, a rectal cancer, a melanoma, a glioblastoma, or a sarcoma.

In some embodiments of the invention direct administration is to two or more lymph nodes or lymph vessels. In some embodiments the lymph node is selected from the group consisting of, for example, inguinal, axillary, cervical, and tonsilar lymph nodes, and the like.

In some embodiments of the invention the CTL response is specific for the first antigen. In some embodiments the epitope is a housekeeping epitope. In some embodiments the first and second compositions include a carrier suitable for direct administration to the lymphatic system or a lymph node or the like. In some embodiments of the invention the epitope is an immune epitope. In some embodiments the delivering substep or the administering substep includes a single bolus injection. In some embodiments the delivering substep or the administering substep includes repeated bolus injections. In some embodiments the delivering substep or the administering substep includes a continuous infusion.

In some embodiments of the invention the chemotherapeutic agent downregulates or depletes T-regulatory cell activity thereby promoting or enhancing effector T cell activity within, for example, a tumor or cancer cell or the like. In some embodiments, interfering with T-regulatory cell function includes, for example, a reduction in the number of T-regulatory cells. In some embodiments, the reduction in number of T-regulatory cells is measured using flow cytometry. In some embodiments the reduction in number of T-regulatory cells is measured using markers such as, for example CD4+, CD25+,FoxP3HI, or the like.

In some embodiments of the invention, interfering with T-regulatory cell function includes impairing the activity of T-regulatory cells. In some embodiments, the activity of T-regulatory cells is measured, for example, by isolating T-regulatory cells from the patient, incubating the isolated cells with effector cells in a standard assay of effector cell function, and measuring effector cell activity. In some embodiments, the standard assay of effector cell function is selected from the group consisting of: a CTL assay, an elispot assay, and a proliferation assay. In some embodiments, the effector T cell response can be detected by at least one indicator for example, a cytokine assay, an Elispot assay, a cytotoxicity assay, a tetramer assay, a DTH-response, a clinical response, tumor shrinkage, tumor clearance, inhibition of tumor progression, decrease pathogen titer, pathogen clearance, amelioration of a disease symptom, and the like.

In some embodiments of the invention, the chemotherapeutic agent is selected from the group including, for example, cyclophosphamide, gemcitabine, fludarabine, doxorubicin, and the like. In some embodiments the chemotherapeutic agent is cyclophosphamide, the contacting step is performed upon observation of rising T-regulatory cell function, or induction of abnormal cell proliferation, or tumor growth. In some embodiments, the contacting and inducing steps are repeated in two or more cycles. In some embodiments the contacting and inducing steps are repeated until, for example, a reduction in T-regulatory cell activity or a regression of abnormal cell proliferation or tumor growth, or the like, is achieved.

In some embodiments of the invention, the contacting step precedes the inducing step. In some embodiments the contacting step is repeated prior to the inducing step. In some embodiments the contacting step is completed about one week prior to the inducing step. In some embodiments, the contact step is completed 6, 7, 8, or 9 days prior to the inducing step. In some embodiments the contacting step is repeated prior to the administering substep of the inducing step. In some embodiments the delivering substep and the administering substep are carried out on different days. In some embodiments the delivering substep and the administering substep are carried out on at least about 2, 3, 4, 5, 6, or 7 days apart.

In some embodiments of the invention the delivering substep of the inducing step occurs after the contacting step. In some embodiments the delivering substep includes administering one or more peptides corresponding to an epitope of the antigen prior to or after administering a chemotherapeutic agent.

Some embodiments of the invention also include administering at least one mode of treatment, for example radiation therapy, gene therapy, biochemotherapy, surgery, and the like, in addition to the combination chemotherapeutic/immunotherapeutic regimen. In some embodiments the at least one mode of treatment is provided prior to or during the contacting step. In some embodiments the at least one mode of treatment is provided prior to the contacting and inducing steps. In some embodiments, the at least one mode of treatment is completed prior to commencing the contacting and inducing steps of the chemotherapeutic/immunotherapeutic regimen. Thus, in some embodiments, complete remission is attained prior to commencing the contacting and inducing steps. In other embodiments, complete remission is not necessarily attained prior to commencing the combination chemotherapeutic/immunotherapeutic regimen. In one embodiment, the at least one mode of treatment is administered after one, two, or more complete cycles of the contacting and inducing step of the chemotherapeutic/immunotherapeutic regimen. In another embodiment, the at least one mode of treatment is administered in conjunction with the contacting and inducing steps of the chemotherapeutic/immunotherapeutic regimen.

The antigen can be a disease-associated antigen, and the disease-associated antigen can be a tumor-associated antigen, or a pathogen-associated antigen. Embodiments include methods of treating a disease, such as cancer, utilizing the described method of immunizing. An antigen as contemplated herein can be a target-associated antigen. The target can be a neoplastic cell, a pathogen-infected cell, and the like. For example, any neoplastic cell can be targeted. Pathogen-infected cells can include, for example, cells infected by a bacterium, a virus, a protozoan, a fungus, and the like, or affected by a prion, for example.

Some embodiments of the invention are directed toward the use of a chemotherapeutic agent and a CTL inducing combination medicament in the manufacture of an immunizing combination medicament, where the chemotherapeutic agent achieves at least one of, for example, promoting tumoral inflammation and interfering with T-regulatory cell function; and where the CTL combination medicament includes a first composition for delivering to a patient, and the first composition includes an immunogen, and the immunogen includes or encodes for at least part of a first antigen or an immunogenic fragment thereof; and a second composition for administering directly to a lymphatic system of the patient, with the second composition including a peptide, and the peptide corresponds to an epitope of the first antigen; and where the combination results an enhanced effectiveness of treatment beyond the effectiveness of either of the chemotherapeutic agent or the CTL inducing combination medicament alone.

Further embodiments can include sets of immunogenic compositions for inducing a class I MHC-restricted immune response in a patient including 1-6 entraining doses and at least one amplifying dose, wherein the entraining doses can include an immunogen or a nucleic acid encoding an immunogen, and wherein the amplifying dose can include a peptide epitope, and wherein the epitope can be presented by pAPC, and wherein the sets further include, or are for use with, a chemotherapeutic agent. The nucleic acid encoding the immunogen further can include an immunostimulatory sequence which can be capable of functioning as the immunopotentiating agent. The immunogen can be a virus or replication-competent vector that can include or can induce an immunopotentiating agent. The immunogen can be a bacterium, bacterial lysate, or purified cell wall component. Also, the bacterial cell wall component can be capable of functioning as the immunopotentiating agent. The immunopotentiating agent can be, for example, a TLR ligand, an immunostimulatory sequence, a CpG-containing DNA, a dsRNA, an endocytic-Pattern Recognition Receptor (PRR) ligand, an LPS, a quillaja saponin, tucaresol, a pro-inflammatory cytokine, and the like. In some preferred embodiments for promoting multivalent responses the sets can include multiple entraining doses and/or multiple amplification doses corresponding to various individual antigens, or combinations of antigens, for each administration. The multiple entrainment doses can be administered as part of a single composition or as part of more than one composition. The sets can optionally include at least one chemotherapeutic agent.

The amplifying doses can be administered at disparate times and/or to more than one site, for example. The chemotherapeutic agent can be administered prior to, during, or after any of the entraining doses and/or the amplifying doses. In some embodiments, the chemotherapeutic agent is administered after initiation of the immunotherapeutic protocol.

An amplifying peptide used in the various embodiments corresponds to an epitope of the immunizing antigen. In some embodiments, correspondence can include faithfully iterating the native sequence of the epitope. In some embodiments, correspondence can include the corresponding sequence can be an analogue of the native sequence in which one or more of the amino acids have been modified or replaced, or the length of the epitope altered. Such analogues can retain the immunologic function of the epitope (i.e., they are functionally similar). In particular embodiments the analogue has similar or improved binding with one or more class I MHC molecules compared to the native sequence. In other embodiments the analogue has similar or improved immunogenicity compared to the native sequence. Strategies for making analogues are widely known in the art. Exemplary discussions of such strategies can be found in U.S. patent application Ser. No. 10/117,937 (Pub. No. 2003-0220239 A1), filed on Apr. 4, 2002; and Ser. No. 10/657,022 (Publication No. 20040180354), filed on Sep. 5, 2003, both entitled EPITOPE SEQUENCES; and U.S. Provisional Patent Application No. 60/581,001, filed on Jun. 17, 2004 and U.S. patent application Ser. No. 11/156,253 (Pub. No. 2006-0063913), filed on Jun. 17, 2005, both entitled SSX-2 PEPTIDE ANALOGS; and U.S. Provisional Patent Application No. 60/580,962 and U.S. patent application Ser. No. 11/155,929 (Pub. No. 20060094661), filed on Jun. 17, 2005, both entitled NY-ESO PEPTIDE ANALOGS; each of which is hereby incorporated by reference in its entirety.

Some embodiments relate to uses of a peptide in the manufacture of an adjuvant-free medicament for use in an entrain-and-amplify immunotherapy/chemotherapeutic combination protocol. The compositions, kits, immunogens and compounds can be used in medicaments for the treatment of various diseases such as but not limited to cancer, to amplify immune responses, to generate particular cytokine profiles, and the like, as described herein. Embodiments relate to the use of adjuvant-free peptide in a method of amplifying an immune response.

In some embodiments, the combination immunotherapeutic/chemotherapeutic strategies disclosed herein include methods, uses, therapies and compositions related to epitopes with specificity for MHC, including, for example, as disclosed in U.S. Provisional Application No. 60/640,402, filed on Dec. 29, 2004, and U.S. application Ser. No. 11/323,572 (Pub. No. 20060165711), filed on Dec. 29, 2005, all of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES”. Other embodiments include one or more of the MHCs as disclosed in U.S. Provisional Application No. 60/640,402, filed on Dec. 29, 2004, and U.S. application Ser. No. 11/323,572 (Pub. No. 20060165711), filed on Dec. 29, 2005, all of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES”, including combinations of the same, while other embodiments specifically exclude any one or more of the MHCs or combinations thereof. U.S. Provisional Application No. 60/640,402, filed on Dec. 29, 2004, and U.S. application Ser. No. 11/323,572 (Pub. No. 20060165711), filed on Dec. 29, 2005, all of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES” (each of which is incorporated herein by reference in its entirety) include frequencies for the listed HLA antigens.

Various antigen combinations are provided in U.S. application Ser. No. 10/871,708 (Pub. No. 20050118186), filed on Jun. 17, 2004, entitled COMBINATIONS OF TUMOR-ASSOCIATED ANTIGENS IN COMPOSITIONS FOR VARIOUS TYPES OF CANCERS; and U.S. Provisional Application No. 60/640,598, filed on Dec. 29, 2004, and in U.S. application Ser. No. 11/323049 (Pub. No. 20060159694), filed on Dec. 29, 2005, both also entitled COMBINATIONS OF TUMOR-ASSOCIATED ANTIGENS IN COMPOSITIONS FOR VARIOUS TYPES OF CANCERS, each of which is incorporated herein by reference in its entirety. Preferably the antigen, including antigen A or B can be SSX-2, Melan-A, Tyrosinase, PSMA, PRAME, NY-ESO-1, or the like. Many other antigens are known to those of ordinary skill in the art. It should be understood that in this and other embodiments, more than two compositions, immunogens, antigens, epitopes and/or peptides can be used. For example, three, four, five or more of any one or more of the above can be used.

In combination with the immunotherapeutic/chemotherapeutic strategy disclosed herein, other therapeutic strategies can also be employed. For example, the combination immunotherapeutic/chemotherapeutic strategy can be used in combination with, for example, but not limited to, radiotherapy, biotherapy, gene therapy, hormonal therapy, or surgery, and the like.

Therefore, the present invention provides a method of treating a subject having a cancer or tumor comprising providing an immunotherapeutic regimen in combination with a chemotherapeutic composition further combined with at least one mode of treatment selected from the group of radiation therapy, chemotherapy, gene therapy biochemotherapy, and surgery.

Combination of immunotherapeutic/chemotherapeutic strategies, as disclosed herein, with additional treatment modalities can increase the susceptibility of tumoral processes to the elicited immune response and thereby result in increased therapeutic benefit. In some embodiments, the therapeutic benefit is synergistically enhanced. Tumor debulking prior to or during immunotherapy/chemotherapy increases the potential for any particular level of immune response to slow or halt disease progression or to bring about tumor regression or elimination. Additionally, tissue damage, necrosis, or apoptosis initiated with antibody therapy, radiotherapy, biotherapy, chemotherapy, passive immunotherapy (including treatment with mono- and/or polyclonal antibodies, recombinant TCR, and/or adoptive transfer of CTL or other cells of the immune system, or activators of the inate immune system such as CpG oligonucleotides and other TLR ligands) or surgery, can facilitate the immunotherapeutic/chemotherapeutic approach via general inflammation resulting in recruitment of immune effector cells including antigen-specific effectors. In general, any method to induce a transient or more permanent general inflammation within one or multiple tumors/metastatic lesions can facilitate the active immunotherapy. Alternatively or in addition to enabling recruitment of effectors, general inflammation can also increase the susceptibility of target cells to immune mediated attack (e.g., as interferons increase expression of target molecules on cancer cells and underlying stroma).

In preferred embodiments, delivering the immunotherapeutic can include direct administration to the lymphatic system of the patient. The direct administration to the lymphatic system of the patient can include direct administration to a lymph node or lymph vessel. The direct administration can be to two or more lymph nodes or lymph vessels. The lymph node can be, for example, inguinal, axillary, cervical, and tonsilar lymph nodes.

In some embodiments, delivering or administering the immunotherapeutic can include delivering as a single bolus injection or repeated bolus injections, for example. In some embodiments, delivering or administering the immunotherapeutic can include a continuous infusion, which for example, can have duration of between about 8 to about 7 days. The method can include an interval between termination of the delivering step and beginning the administering step, wherein the interval can be at least about seven days. Also, the interval can be between about 7 and about 14 days, about 17 days, about 20 days, about 25 days, about 30 days, about 40 days, about 50 days, or about 60 days, for example. The interval can be over about 75 days, about 80 days, about 90 days, about 100 days or more.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts tumor protection in mice prophylactically immunized with E7₄₉₋₅₇ peptide from HPV16.

FIG. 2 illustrates substantial regression of tumors in mice therapeutically immunized with E7₄₉₋₅₇ peptide from HPV16 on days 7, 10, 21, and 24 following tumor challenge as compared to the control group (p<0.0001).

FIG. 3 shows a correlation of the immune response with that of tumor eradication in cured mice versus relapsing mice immunized with E7₄₉₋₅₇ peptide from HPV16 (p=0.04).

FIG. 4 shows that relapsing mice immunized with an additional boost of E7₄₉₋₅₇ peptide showed a significant immune response but no measurable increase in tumor efficacy.

FIG. 5 shows a large percentage of antigen specific tumor infiltrating lymphocytes (TILs) in mice immunized with E7₄₉₋₅₇ peptide from HPV16 as compared to the control mice group.

FIG. 6 depicts an increase in the number of CD4⁺CD25⁺Fox P3⁺ T-regulatory cells in tumor bearing mice (Panel B) compared to naïve (Panel A), cured (Panel D), and cyclophosamide (100 mg/kg) injected mice (Panels C). Panel E shows the average percentage of T-regulatory cells in the spleen of mice from Panels A-D.

FIG. 7 depicts the immuno-modulatory effects of combining the E7₄₉₋₅₇ peptide immunotherapeutic regimen and cyclophosphamide.

FIG. 8 depicts the immunological protection from disseminated disease in mice injected with HPV-16 peptide or HPV-16 peptide and dsRNA (polyIC). Panel A shows Tetramer staining on Day 25 from peripheral blood. Panel B shows the percent survival for each group of mice.

FIG. 9 depicts the anti-tumor efficacy of intranodal versus conventional dosing of HPV-16. Panel A shows the tumor size for each group. Panel B shows Tetramer staining on Day 31 from peripheral blood.

FIG. 10 depicts the reduction in the level of T-regs in mice bearing HPV-16 transformed tumors in the presence of cyclophosphamide. Panel A and Panel B show the reduction of T-regs in spleen. Panel C shows the reduction of T-regs in tumor.

FIG. 11 depicts the efficacy of adjunctive therapy in late stage cancer. Panel A shows tumor progression in the presence of cyclophosphamide or E7₄₉₋₅₇ immunotherapy, or the combination of cyclophosphamide and E7₄₉₋₅₇ immunotherapy. Panel B shows the immune response in mice treated with cyclophosphamide or E7₄₉₋₅₇ immunotherapy, or the combination of cyclophosphamide and E7₄₉₋₅₇ immunotherapy.

FIG. 12 depicts the effect of adjunctive therapy on survival in mice treated with chemotherapy and immunotherapy.

FIG. 13 depicts subcutaneous immunotherapy dosing arm and tumor efficacy resulting from subcutaneous versus intra-lymphatic immunotherapy.

FIG. 14 depicts adjuvant efficacy, showing that active immunotherapy improves progression free survival and time to relapse post primary tumor removal, by chemotherapy or surgery.

FIG. 15 depicts neoadjuvant efficacy, showing that active immunotherapy improves the rate of response and showing clinical benefit when applied prior to primary tumor treatment, by chemotherapy or surgery.

FIG. 16 depicts consolidation therapy, showing that active immunotherapy improves progression free survival and time to progression post chemotherapy.

FIG. 17 depicts adjunctive therapy, showing that active immunotherapy improves the rate of response when it accompanies surgery or chemotherapy.

### DETAILED DESCRIPTION OF THE INVENTION

Previous immunization protocols have shown a reduced production of T-regulatory cells. However, previously, it was not known whether it would be possible to enhance the effectiveness of an immune response by further depleting T-regulatory cells. For example, it was not known whether further depletion would have any additional effect on the immune response. Likewise, it was not known whether use of a chemotherapeutic agent would have a negative impact on cytotoxic T lymphocyte (CTL) activation and function, that would offset any potential benefit of T-regulatory cell depletion. Herein is reported the unexpected result that chemotherapeutic agents that downregulate or deplete T-regulatory cells can be used in conjunction with “entrain and amplify” immunotherapeutic protocols with enhanced results.

A two-stage immunization protocol for the generation of a robust CTL response has previously been described. See U.S. Provisional Application No. 60/479,393, filed on Jun. 17, 2003, entitled METHODS TO CONTROL MHC CLASS I-RESTRICTED IMMUNE RESPONSE; U.S. application Ser. No. 10/871,707 filed on Jun. 17, 2004 (Pub. No. 20050079152), U.S. Provisional Application No. 60/640,402, filed on Dec. 29, 2004, and U.S. application Ser. No. 11/323,572 (Pub. No. 20060165711), filed on Dec. 29, 2005, all three of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES.” Each of the applications, including all methods, figures, and compositions, is incorporated herein by reference in its entirety. The initiating stage, referred to as induction or entrainment, includes immunization against a target antigen so as to induce at least a minimal response to at least one CTL epitope. In preferred embodiments it includes an immunopotentiating agent to entrain an effector response. In a preferred embodiment, this is accomplished by intranodal administration of 1) a plasmid causing expression of the CTL epitope and having a CpG immunostimulatory sequence, or 2) an epitopic peptide and an immunopotentiator such as dsRNA or a CpG oligonucleotide. However in other embodiments it is possible to use more traditional compositions and routes of administration. The initiation stage can include a single bolus injection, multiple injections within a few days of each other, or continuous infusion for several (e.g. 3-7) days. Such a course can be repeated at intervals, typically of 1 to 3 weeks, typically for a total of 2 or 3 courses, but more courses, or just a single course, are also possible.

In the second stage of the immunization protocol, referred to as amplification, an epitopic peptide corresponding to the CTL epitope against which a response was induced in the first stage is administered to the lymphatic system, preferably intranodally. It is not necessary to include an immunopotentiator or other adjuvant, although one can be present in some embodiments. For example, epitopic peptide plus dsRNA can be used as both an entraining and an amplifying composition. The schedule and mode of administration can be similar to that described above for the initiation stage, however, typically somewhat more courses (2 to 4 or more rather than 1 to 3 or more) are administered and the interval between courses, as well as between the stages, can be 1 to 3 or more weeks extending to several months. A course of inducing doses followed by a course of amplifying doses is referred to as a therapeutic cycle. Treatment will generally involve multiple therapeutic cycles.

It was found that by using these particular compositions in the above-described order (the entrain-and-amplify immunization protocol) it was possible to generate large numbers of antigen specific CD8+ T cells with stable effector (e.g., CTL) phenotype. This was in contrast to alternative protocols. For example intranodal administration of epitopic peptide can generate a cytotoxic/cytolytic T cell (CTL) response, attempts to further amplify this response with further injections can lead to the expansion of a regulatory T cell population and a diminution of observable CTL activity. The design, practice and effects of such immunization protocols are fully described in U.S. Provisional Application No. 60/479,393, filed on Jun. 17, 2003, entitled METHODS TO CONTROL MHC CLASS I-RESTRICTED IMMUNE RESPONSE; U.S. application Ser. No. 10/871,707 filed on Jun. 17, 2004 (Pub. No. 20050079152), U.S. Provisional Application No. 60/640,402, filed on Dec. 29, 2004, and U.S. application Ser. No. 11/323,572 (Pub. No. 20060165711), filed on Dec. 29, 2005, all three of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES” each of which are hereby incorporated by reference in their entirety.

The tumor environment is often refractory to immunological attack. It is desirable in cancer immunotherapy to make the tumor environment less refractory so as to increase the activity of CTL or other effector T cells within the tumor and to improve the overall efficacy of treatment. As used herein, “efficacy” refers to the ability of a chemotherapeutic and/or immunogenic composition or of a combination treatment to achieve a desired action or result. One possible approach is to combine immunotherapy with use of chemotherapeutic agents that deplete or down-regulate regulatory T cells (Treg) or that increase the pro-inflammatory nature of the tumor environment. Traditionally, active immunotherapy and chemotherapy have been separated in time to avoid impairing or preventing the immune response. Moreover, as the immunization protocol above generates reduced numbers of Treg cells it was not clear that it could be improved by further depletion of this population. It has now been found that it is indeed possible to combine an entrain-and-amplify immunization protocol with use of a chemotherapeutic agent such that the overall effectiveness of the combined treatment is greater than the effectiveness of the chemotherapeutic or the entrain-and-amplify immunization protocol alone. Indeed the combination was synergistic as substantial tumor regression was obtained under conditions in which either treatment alone had no effect on tumor growth.

In other embodiments of the invention the combination immunotherapy/chemotherapy protocol can incorporated into standard oncology therapy paradigms such as Adjunctive or Consolidation Therapy, involving surgery, radiation, or higher doses of chemotherapy, and the like.

In other embodiments of the invention the combination immunotherapy/chemotherapy protocol can incorporated into standard oncology therapy paradigms such as Adjunctive or Consolidation Therapy, involving surgery, radiation, or higher doses of chemotherapy, and the like.

In combining chemotherapy and immunotherapy, the dose of chemotherapeutic agent chosen by the practitioner can generally be less than that used for direct cytotoxicity against the tumor cells, but great enough to be lymphocytotoxic. In some embodiments, the chemotherapeutic agent can impair the function of Treg cells without necessarily depleting them. Such treatment can impair, whether by depletion or deactivation, the functionality of Treg cells resident in the tumor, thereby making the tumor environment less refractory to effector T cells, such as CTL. Additionally, although the dosage of chemotherapeutic agent used is insufficient to shrink tumors or halt their growth, there can still be cellular damage contributing to a more pro-inflammatory environment within the tumor, thereby promoting the recruitment and activity of effector T cells.

In some embodiments, the chemotherapeutic agent is administered in the week prior to initiating immunization. As the Treg resident in the tumor are depleted and the immunization protocol is biased against the generation of Treg, a robust effector response is obtained and tumor shrinkage or eradication is observed. In other embodiments of the invention, the chemotherapeutic agent is administered in the interval between the induction stage and the amplification stage, between courses of the amplifying composition, or between therapeutic cycles. In preferred embodiments of each of these cases, chemotherapy is initiated approximately a week (6, 7, 8, or 9 days) prior to beginning the next course of immunization. If multiple doses of the chemotherapeutic agent are to be given it is generally preferred that that last dose be given 0, 1, or 2 days prior to beginning the next course of immunization.

In various embodiments the above, combination therapy is carried out in various relations to other cancer therapies. It can be used in an adjuvant setting to increase the likelihood of a cure. That is, the cancer can be put into complete remission by a tumor ablative treatment such as, for example, but not limited to, surgical removal, irradiation, or chemotherapy with doses that are directly cytotoxic to the cancer cells, and the like. The combination therapy is subsequently undertaken, resulting in a decreased rate of relapse and increased interval of disease-free survival. In various embodiments it is preferred that the combination protocol commence within four days, one week, or two weeks of the completion of the initial treatment. In some but not all embodiments involving direct chemotherapy as the initial treatment, no additional administration of the chemotherapeutic agent is required and it is the immunization portion of the combination therapy that commences within the stated interval.

In other embodiments, generally with less bulky disease, the combination therapy can be used in a neoadjuvant setting. That is, at least one therapeutic cycle of the combination therapy is completed prior to a tumor ablative treatment such as, for example, but not limited to, surgery, radiation, or direct chemotherapy. In various embodiments, the tumor ablative treatment is commenced within four days, one week, or two weeks of the completion of the therapeutic cycle. These patients display an increased rate of complete and partial remission and a decreased rate of relapse at the same site or a remote site, plus an increased median disease free survival.

In still other embodiments the combination therapy is used as consolidation therapy. This resembles the adjuvant setting above except that complete remission is not necessarily attained. The combination therapy produces an increased time to progression, and progression-free survival (in the case of partial remission) and increased time to relapse (in the case of complete remission).

In yet other embodiments the combination therapy can be used as adjunctive therapy, that is, in further combination with a tumor ablative treatment to increase that treatment's efficacy. In contrast to adjuvant therapy as described above in which the combination therapy is not initiated until the primary treatment is complete, here the two treatments are used together to increase the rate of response (that is of partial or complete remission). The actual schedule of the two treatments can be similar to those above, but therapeutic cycles of the combination therapy can be alternated with rounds of the primary treatment such as chemotherapy or radiation. In alternative embodiments, surgery can be carried out during the time interval of a therapeutic cycle of the combination therapy, preferably in the interval between the induction and amplification stages or in an interval between courses of the amplification composition.

Embodiments of the invention disclosed herein provide a novel approach to overcome the deficiencies in the art by targeting APC in situ through intra-lymphatic administration of plasmids designed to prime an anti-tumor CTL response, followed by boosting with peptide epitopes to dramatically expand and activate the pool of antigen specific T cells, wherein a chemotherapeutic agent is administered prior to, during, or after the targeting or boosting steps. In a particular embodiment, the chemotherapeutic agent is cyclophosphamide.

Some embodiments provide methods and compositions, for example, for generating immune cells specific to a target cell, for directing an effective immune response against a target cell, or for affecting/treating proliferative cell disorders. Proliferative cell disorders include for example, cancers or tumors such as, but not limited to, those of the prostate, ovary, breast, skin, lung, or kidney.

The methods and compositions can include, for example, immunogenic compositions such as vaccines and therapeutics, and also prophylactic and therapeutic methods. By selecting the form of antigen, the sequence and timing with which it is administered, and delivering the antigen directly into secondary lymphoid organs, not only the magnitude, but the qualitative nature of the immune response can be managed, and that combining this approach with additional therapeutic strategies such as chemotherapy, enhances the efficacy of treatment.

Some preferred embodiments relate to compositions and methods for entraining and amplifying a T cell response for use in combination with a chemotherapeutic agent. For example such methods can include an entrainment step wherein a composition containing a nucleic acid encoded immunogen is delivered to an animal. The composition can be delivered to various locations on the animal, but preferably is delivered to the lymphatic system, for example, a lymph node or an area of lymphatic drainage. The entrainment step can include one or more deliveries of the composition, for example, spread out over a period of time or in a continuous fashion over a period of time. Preferably, the methods can further include an amplification step comprising administering a composition containing an epitopic peptide immunogen. The amplification step can be performed one or more times, for example, at intervals over a period of time, in one bolus, or continuously over a period of time. Although not required in all embodiments, some embodiments of the amplification step can include the use of compositions that include an immunopotentiator or adjuvant. The chemotherapeutic agent can be administered prior to, during, or after either an entrainment or amplifying dose. In one embodiment, prior to or after an entrainment dose.

Each of the disclosures of the following applications, including all methods, figures, and compositions, is incorporated herein by reference in its entirety: U.S. Provisional Application No. 60/479,393, filed on Jun. 17, 2003, entitled METHODS TO CONTROL MHC CLASS I-RESTRICTED IMMUNE RESPONSE; U.S. application Ser. No. 10/871,707 filed on Jun. 17, 2004 (Pub. No. 20050079152), U.S. Provisional Application No. 60/640,402, filed on Dec. 29, 2004, and U.S. application Ser. No. 11/323,572 (Pub. No. 20060165711), filed on Dec. 29, 2005, all three of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES“; U.S. application Ser. No. 10/871,708 (Pub. No. 20050118186), filed on Jun. 17, 2004, entitled “COMBINATIONS OF TUMOR-ASSOCIATED ANTIGENS IN COMPOSITIONS FOR VARIOUS TYPES OF CANCERS”; and Provisional Application No. 60/640,598, filed on Dec. 29, 2004, and U.S. patent application Ser. No. 11/323,049 (Pub. No. 20060159694), filed on Dec. 29. 2005, both of which are entitled “COMBINATIONS OF TUMOR-ASSOCIATED ANTIGENS IN COMPOSITIONS FOR VARIOUS TYPES OF CANCERS,” and each of which are incorporated by reference in its entirety. Also, the following applications include methods and compositions that can be used with the instant methods and compositions. Plasmid and principles of plasmid design are disclosed in U.S. patent application Ser. No. 10/292,413 (Pub. No. 20030228634 A1), entitled “EXPRESSION VECTORS ENCODING EPITOPES OF TARGET ASSOCIATED ANTIGENS AND METHODS FOR THEIR DESIGN,” which is hereby incorporated by reference in its entirety; additional methodology, compositions, peptides, and peptide analogues are disclosed in U.S. Provisional Application No. 60/581,001, filed on Jun. 17, 2004, U.S. application Ser. No. 11/156,253 (Pub. No. 20060063913), entitled “SSX-2 PEPTIDE ANALOGS”; each of which is incorporated herein by reference in its entirety; U.S. Provisional Application No. 60/580,962, filed on Jun. 17, 2004, U.S. application Ser. No. 11/155,929 (Pub. No. 20060094661), filed on Jun. 17, 2005, entitled “NY-ESO PEPTIDE ANALOGS”; each of which is incorporated herein by reference in its entirety; and U.S. application Ser. No. 10/117,937 (Pub. No. 20030220239), filed on Apr. 4, 2002, and Ser. No. 10/657,022 (Pub. No. 20040180354), filed on Sep. 5, 2003, both of which are entitled EPITOPE SEQUENCES, and each of which is hereby incorporated by reference in its entirety.

In some embodiments, depending on the nature of the immunogen and the context in which it is encountered, the immune response elicited can differ in its particular activity and makeup. In particular, while immunization with peptide can generate a cytotoxic/cytolytic T cell (CTL) response, attempts to further amplify this response with further injections can instead lead to the expansion of a regulatory T cell population, and a diminution of observable CTL activity. Thus, compositions conferring high MHC/peptide concentrations on the cell surface within the lymph node, without additional immunopotentiating activity, can be used to purposefully promote a regulatory or tolerogenic response. In contrast, immunogenic compositions providing ample immunomodulating signals (e.g., toll-like receptor ligands, or the cytokine/autocrine factors such ligands can induce) even if providing only limiting antigen, not only induce a response, but entrain it as well, so that subsequent encounters with ample antigen (e.g., injected peptide) amplifies the response without changing the nature of the observed activity. Therefore, some embodiments relate to controlling the immune response profile, for example, the kind of response obtained and the kinds of cytokines produced. Some embodiments relate to methods and compositions for promoting the expansion or further expansion of CTL.

The disclosed methods are advantageous over many protocols that use only peptide or that do not follow the entrain-and-amplify methodology. As set forth above, many peptide-based immunization protocols and vector-based protocols have drawback CTL response potentiation by up-regulation of Treg response. Nevertheless, if successful, a peptide based immunization or immune amplification strategy has advantages over other methods, particularly certain microbial vectors, for example. This is due to the fact that more complex vectors, such as live attenuated viral or bacterial vectors, can induce deleterious side-effects, for example, in vivo replication or recombination; or become ineffective upon repeated administration due to generation of neutralizing antibodies against the vector itself. Additionally, when harnessed in such a way as to become strong immunogens, peptides can circumvent the need for proteasome-mediated processing (as with protein or more complex antigens, in context of “cross-processing” or subsequent to cellular infection). That is because peptides resulting from cellular processing of complex antigens for MHC-class I restricted presentation is a phenomenon that inherently selects dominant (favored) epitopes over subdominant epitopes, potentially interfering with the immunogenicity of epitopes corresponding to valid targets. Finally, effective peptide-based immunization simplifies and shortens the process of development of immunotherapeutics.

### DEFINITIONS

Unless otherwise clear from the context of the use of a term herein, the following listed terms shall generally have the indicated meanings for purposes of this description.

PROFESSIONAL ANTIGEN-PRESENTING CELL (pAPC)—a cell that possesses T cell costimulatory molecules and is able to induce a T cell response. Well characterized pAPCs include dendritic cells, B cells, and macrophages.

PERIPHERAL CELL—a cell that is not a pAPC.

HOUSEKEEPING PROTEASOME—a proteasome normally active in peripheral cells, and generally not present or not strongly active in pAPCs.

IMMUNOPROTEASOME—a proteasome normally active in pAPCs; the immunoproteasome is also active in some peripheral cells in infected tissues or following exposure to interferon.

EPITOPE—a site on an antigen recognized by an antibody or an antigen receptor. A T-cell epitope is a short peptide derived from a protein antigen. Epitopes bind to MHC molecules and are recognized by a particular T cell. In preferred embodiments, epitopes according to this definition include, but are not necessarily limited to, a polypeptide and a nucleic acid encoding a polypeptide, wherein the polypeptide is capable of stimulating an immune response. In other preferred embodiments, epitopes according to this definition include but are not necessarily limited to peptides presented on the surface of cells, the peptides being non-covalently bound to the binding cleft of class I MHC, such that they can interact with T cell receptors (TCR). Epitopes presented by class I MHC can be in immature or mature form. “Mature” refers to an MHC epitope in distinction to any precursor (“immature”) that can include or consist essentially of a housekeeping epitope, but also includes other sequences in a primary translation product that are removed by processing, including without limitation, alone or in any combination, proteasomal digestion, N-terminal trimming, or the action of exogenous enzymatic activities. Thus, a mature epitope can be provided embedded in a somewhat longer polypeptide, the immunological potential of which is due, at least in part, to the embedded epitope; likewise, the mature epitope can be provided in its ultimate form that can bind in the MHC binding cleft to be recognized by TCR.

MHC EPITOPE—a polypeptide having a known or predicted binding affinity for a patientian class I or class II major histocompatibility complex (MHC) molecule. Some particularly well characterized class I MHC molecules are presented in U.S. Provisional Application No. 60/640,402, filed on Dec. 29, 2004, and U.S. application Ser. No. 11/323,572 (Pub. No. 20060165711), filed on Dec. 29, 2005, all of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES.”

HOUSEKEEPING EPITOPE—In a preferred embodiment, a housekeeping epitope is defined as a polypeptide fragment that is an MHC epitope, and that is displayed on a cell in which housekeeping proteasomes are predominantly active. In another preferred embodiment, a housekeeping epitope is defined as a polypeptide containing a housekeeping epitope according to the foregoing definition, that is flanked by one to several additional amino acids. In another preferred embodiment, a housekeeping epitope is defined as a nucleic acid that encodes a housekeeping epitope according to the foregoing definitions. Exemplary housekeeping epitopes are provided in U.S. patent application Ser. No. 10/117,937, filed on Apr. 4, 2002 (Pub. No. 20030220239 A1), Ser. No. 11/067,159 (Pub. No. 20050221440 A1), filed Feb. 25, 2005, Ser. No. 11/067,064 (Pub. No. 20050142144 A1), filed Feb. 25, 2005, and Ser. No. 10/657,022 (Pub. No. 20040180354 A1), filed Sep. 5, 2003, and in PCT Application No. PCT/US2003/027706 (Pub. No. WO 2004/022709 A2), filed Sep. 5, 2003; and U.S. Provisional Application No. 60/282,211, filed on Apr. 6, 2001; 60/337,017, filed on Nov. 7, 2001; 60/363,210 filed Mar. 7, 2002; and 60/409,123, filed on Sep. 6, 2002. Each of the listed applications is entitled EPITOPE SEQUENCES. Each of the applications mentioned in this paragraph is incorporated herein by reference in its entirety.

IMMUNE EPITOPE—In a preferred embodiment, an immune epitope is defined as a polypeptide fragment that is an MHC epitope, and that is displayed on a cell in which immunoproteasomes are predominantly active. In another preferred embodiment, an immune epitope is defined as a polypeptide containing an immune epitope according to the foregoing definition that is flanked by one to several additional amino acids. In another preferred embodiment, an immune epitope is defined as a polypeptide including an epitope cluster sequence, having at least two polypeptide sequences having a known or predicted affinity for a class I MHC. In yet another preferred embodiment, an immune epitope is defined as a nucleic acid that encodes an immune epitope according to any of the foregoing definitions.

TARGET CELL—In a preferred embodiment, a target cells is a cell associated with a pathogenic condition that can be acted upon by the components of the immune system, for example, a cell infected with a virus or other intracellular parasite, or a neoplastic cell. In another embodiment, a target cell is a cell to be targeted by the vaccines and methods of the invention. Examples of target cells according to this definition include but are not necessarily limited to: a neoplastic cell and a cell harboring an intracellular parasite, such as, for example, a virus, a bacterium, or a protozoan. Target cells can also include cells that are targeted by CTL as a part of an assay to determine or confirm proper epitope liberation and processing by a cell expressing immunoproteasome, to determine T cell specificity or immunogenicity for a desired epitope. Such cells can be transformed to express the liberation sequence, or the cells can simply be pulsed with peptide/epitope.

TARGET-ASSOCIATED ANTIGEN (TAA)—a protein or polypeptide present in a target cell.

TUMOR-ASSOCIATED ANTIGENS (TuAA)—a TAA, wherein the target cell is a neoplastic cell.

HLA EPITOPE—a polypeptide having a known or predicted binding affinity for a human class I or class II HLA complex molecule. Particularly well characterized class I HLAs are presented in U.S. Provisional Application No. 60/640,402, filed on Dec. 29, 2004, and U.S. application Ser. No. 11/323,572 (Pub. No. 20060165711), filed on Dec. 29, 2005, all of which are entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES.”

ANTIBODY—a natural immunoglobulin (Ig), poly- or monoclonal, or any molecule composed in whole or in part of an Ig binding domain, whether derived biochemically, or by use of recombinant DNA, or by any other means. Examples include inter alia, F(ab), single chain Fv, and Ig variable region-phage coat protein fusions.

SUBSTANTIAL SIMILARITY—this term is used to refer to sequences that differ from a reference sequence in an inconsequential way as judged by examination of the sequence. Nucleic acid sequences encoding the same amino acid sequence are substantially similar despite differences in degenerate positions or minor differences in length or composition of any non-coding regions. Amino acid sequences differing only by conservative substitution or minor length variations are substantially similar. Additionally, amino acid sequences comprising housekeeping epitopes that differ in the number of N-terminal flanking residues, or immune epitopes and epitope clusters that differ in the number of flanking residues at either terminus, are substantially similar. Nucleic acids that encode substantially similar amino acid sequences are themselves also substantially similar.

FUNCTIONAL SIMILARITY—this term is used to refer to sequences that differ from a reference sequence in an inconsequential way as judged by examination of a biological or biochemical property, although the sequences may not be substantially similar. For example, two nucleic acids can be useful as hybridization probes for the same sequence but encode differing amino acid sequences. Two peptides that induce cross-reactive CTL responses are functionally similar even if they differ by non-conservative amino acid substitutions (and thus may not be within the substantial similarity definition). Pairs of antibodies, or TCRs, that recognize the same epitope can be functionally similar to each other despite whatever structural differences exist. Testing for functional similarity of immunogenicity can be conducted by immunizing with the “altered” antigen and testing the ability of an elicited response, including but not limited to an antibody response, a CTL response, cytokine production, and the like, to recognize the target antigen. Accordingly, two sequences can be designed to differ in certain respects while retaining the same function. Such designed sequence variants of disclosed or claimed sequences are among the embodiments of the present invention.

EXPRESSION CASSETTE—a polynucleotide sequence encoding a polypeptide, operably linked to a promoter and other transcription and translation control elements, including but not limited to enhancers, termination codons, internal ribosome entry sites, and polyadenylation sites. The cassette can also include sequences that facilitate moving it from one host molecule to another.

EMBEDDED EPITOPE—in some embodiments, an embedded epitope is an epitope that is wholly contained within a longer polypeptide; in other embodiments, the term also can include an epitope in which only the N-terminus or the C-terminus is embedded such that the epitope is not wholly in an interior position with respect to the longer polypeptide.

MATURE EPITOPE—a peptide with no additional sequence beyond that present when the epitope is bound in the MHC peptide-binding cleft.

EPITOPE CLUSTER—a polypeptide, or a nucleic acid sequence encoding it, that is a segment of a protein sequence, including a native protein sequence, comprising two or more known or predicted epitopes with binding affinity for a shared MHC restriction element. In preferred embodiments, the density of epitopes within the cluster is greater than the density of all known or predicted epitopes with binding affinity for the shared MHC restriction element within the complete protein sequence. Epitope clusters are disclosed and more fully defined in U.S. patent application Ser. No. 09/561,571, filed Apr. 28, 2000, entitled EPITOPE CLUSTERS, which is incorporated herein by reference in its entirety.

LIBERATION SEQUENCE—a designed or engineered sequence comprising or encoding a housekeeping epitope embedded in a larger sequence that provides a context allowing the housekeeping epitope to be liberated by processing activities including, for example, immunoproteasome activity, N terminal trimming, and/or other processes or activities, alone or in any combination.

CTLp—CTL precursors are T cells that can be induced to exhibit cytolytic activity. Secondary in vitro lytic activity, by which CTLp are generally observed, can arise from any combination of naïve, effector, and memory CTL in vivo.

MEMORY T CELL—A T cell, regardless of its location in the body, that has been previously activated by antigen, but is in a quiescent physiologic state requiring re-exposure to antigen in order to gain effector function. Phenotypically they are generally CD62L-CD44hi CD107α-IFN-γ-LTβ-TNF-α− and is in G₀ of the cell cycle.

EFFECTOR T CELL—A T cell that, upon encountering antigen, readily exhibits effector function. Effector T cells are generally capable of exiting the lymphatic system and entering the immunological periphery. Phenotypically they are generally CD62L-CD44hi CD107α+IGN-γ+LTβ+TNF-α+ and actively cycling.

EFFECTOR FUNCTION—Generally, T cell activation including acquisition of cytolytic activity and/or cytokine secretion.

INDUCING a T cell response—Includes in many embodiments the process of generating a T cell response from naïve, or in some contexts, quiescent cells; activating T cells.

AMPLIFYING A T CELL RESPONSE—Includes in many embodiments a process for increasing the number of cells, the number of activated cells, the level of activity, rate of proliferation, or similar parameter of T cells involved in a specific response.

ENTRAINMENT—Includes in many embodiments an induction that confers particular stability on the immune profile of the induced lineage of T cells. In various embodiments, the term “entrain” can correspond to “induce,” and/or “initiate.”

TOLL-LIKE RECEPTOR (TLR)—Toll-like receptors (TLRs) are a family of pattern recognition receptors that are activated by specific components of microbes and certain host molecules. As part of the innate immune system, they contribute to the first line of defense against many pathogens, but also play a role in adaptive immunity.

TOLL-LIKE RECEPTOR (TLR) LIGAND—Any molecule capable of binding and activating a toll-like receptor. Examples include, without limitation: poly IC—a synthetic, double-stranded RNA know for inducing interferon. The polymer is made of one strand each of polyinosinic acid and polycytidylic acid, double-stranded RNA, unmethylated CpG oligodeoxyribonucleotide or other immunostimulatory sequences (ISSs), lipopolysacharide (LPS), β-glucans, and imidazoquinolines, as well as derivatives and analogues thereof.

IMMUNOPOTENTIATING ADJUVANTS—Adjuvants that activate pAPC or T cells including, for example: TLR ligands, endocytic-Pattern Recognition Receptor (PRR) ligands, quillaja saponins, tucaresol, cytokines, and the like. Some preferred adjuvants are disclosed in Marciani, D. J. Drug Discovery Today 8:934-943, 2003, which is incorporated herein by reference in its entirety.

IMMUNOSTIMULATORY SEQUENCE (ISS)—Generally an oligodeoxyribonucleotide containing an unmethlylated CpG sequence. The CpG can also be embedded in bacterially produced DNA, particularly plasmids. Further embodiments include various analogues; among preferred embodiments are molecules with one or more phosphorothioate bonds or non-physiologic bases.

VACCINE—In preferred embodiments a vaccine can be an immunogenic composition providing or aiding in prevention of disease. In other embodiments, a vaccine is a composition that can provide or aid in a cure of a disease. In still other embodiments, a vaccine composition can provide or aid in amelioration of a disease. Further embodiments of a vaccine immunogenic composition can be used as therapeutic and/or prophylactic agents.

IMMUNIZATION—a process to induce partial or complete protection against a disease. Alternatively, a process to induce or amplify an immune system response to an antigen. In the second definition it can connote a protective immune response, particularly proinflammatory or active immunity, but can also include a regulatory response. Thus in some embodiments immunization is distinguished from tolerization (a process by which the immune system avoids producing proinflammatory or active immunity) while in other embodiments this term includes tolerization.

The major histocompatibility complex and T cell target recognition, as well as Class I and Class II MHC molecules, estimated gene frequencies of HLA-A and HLA-B antigens, and CT genes are described in U.S. patent application Ser. No. 11/323572, (Pub. No. 20060165711), filed Dec. 29, 2005, which is hereby incorporated by reference in its entirety.

Target Antigens for Use in the Present Invention

Embodiments of the present invention provide an immunotherapeutic protocol in combination with a chemotheapeutic strategy in which the immunotherapeutic protocol includes an immunogen for inducing a T cell response in a subject. Such an immunogen contains or encodes an antigen.

Antigens for use in embodiments of the invention can include, in a non-limiting manner, proteins, peptides, polypeptides and derivatives thereof, and can also include non-peptide macromolecules. Antigens, in some instances, can be matched to the specific disease found in the subject being treated to induce a CTL response (also referred to as a cell-mediated immune response), i.e., a cytotoxic reaction by the immune system that results in lysis of the target cells (e.g., the malignant tumor cells or pathogen-infected cells). The present invention also contemplates target-associated antigens. For example, the target can be any neoplastic cell and stromal tumor cells of a cancer, a pathogen-infected cell, and the like. Pathogen-infected cells can include, for example, cells infected by a bacterium, a virus, a protozoan, a fungus, and the like, or affected by a prion, for example.

In some embodiments, antigens can include tumor antigens, such as, which include tumor-specific antigens (TSAs) or tumor-associated antigens (TAAs) as are well known to one of skill in the art. Additional antigens include differentiation antigens, embryonic antigens, cancer-testis antigens, antigens of oncogenes and mutated tumor-suppressor genes, unique tumor antigens resulting from chromosomal translocations, viral antigens, and others that can be apparent presently or in the future to one of skill in the art. Still other antigens include those found in infectious disease organisms, such as structural and non-structural viral proteins. Potential target microbes contemplated in the present invention, include without limitation, hepatitis viruses (e.g., C, B and delta), herpes viruses, HIV, HTLV, HPV, EBV, and the like. In some embodiments, the HPV16 E7₄₉₋₅₇ antigen, which is both a tumor antigen and a viral antigen, is employed.

In other embodiments of the invention, large protein-based antigens can be employed. Such antigens include: differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens can include: TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, β-Catenin, CDK4, Mum-1, p15, p16, 43-9F, 5T4, 791Tgp72, alpha-fetoprotein, β-HCG, BCA225, BTAA, CA 125, CA 15-3\CA 27.29\BCAA, CA 195, CA 242, CA-50, CAM43, CD68\KP1, CO-029, FGF-5, G250, Ga733\EpCAM, HTgp-175, M344, MA-50, MG7-Ag, MOV18, NB/70K, NY-CO-1, RCAS1, SDCCAG16, PLA2, TA-90\Mac-2 binding protein\cyclophilin C-associated protein, TAAL6, TAG72, TLP, and TPS. Protein-based antigens are generally well known to one of ordinary skill in the art.

In other embodiments of the invention, peptide antigens of 8-15 amino acids in length can be employed. Such a peptide can be an epitope of a larger antigen, i.e., it is a peptide having an amino acid sequence corresponding to the site on the larger molecule that is presented by MHC/HLA molecules and can be recognized by, for example, an antigen receptor or T-cell receptor. These smaller peptides are available to one of skill in the art and can be obtained by following the teachings of U.S. Pat. Nos. 5,747,269 and 5,698,396; and PCT Application Numbers PCT/EP95/02593 filed Jul. 4, 1995, and PCT/DE96/00351 filed Feb. 26, 1996, all of which are incorporated herein by reference. Additional approaches to epitope discovery are described in U.S. Pat. Nos. 6,037,135 and 6,861,234, each of which is incorporated herein by reference in its entirety.

Generally, the antigen ultimately recognized by a T cell is a peptide, however, the form of antigen actually administered as the immunogenic preparation need not be a peptide per se. When administered, the epitopic peptide(s) can reside within a longer polypeptide, whether the complete protein antigen, some segment of it, or some engineered sequence. Engineered sequences can include polyepitopes and epitopes incorporated into some carrier sequence such as an antibody or viral capsid protein. Such longer polypeptides can include epitope clusters as described in U.S. patent application Ser. No. 09/561,571 entitled “EPITOPE CLUSTERS,” which is incorporated herein by reference in its entirety. The epitopic peptide, or the longer polypeptide in which it is contained, can be a component of a microorganism (e.g., a virus, bacterium, protozoan, etc.), or a mammalian cell (e.g., a tumor cell or antigen presenting cell), or lysates, whole or partially purified, of any of the foregoing. They can be used as complexes with other proteins, for example heat shock proteins. The epitopic peptide can also be covalently modified, such as by lipidation, or made a component of a synthetic compound, such as dendrimers, multiple antigen peptides systems (MAPS), and polyoximes, or can be incorporated into liposomes or microshperes, etc.

The following discussion sets forth the present understanding or belief of the operation of aspects of the invention. However, it is not intended that this discussion limit the patent to any particular theory of operation not set forth in the claims.

Effective immune-mediated control of tumoral processes or microbial infections generally involves induction and expansion of antigen-specific T cells endowed with multiple capabilities such as migration, effector functions, and differentiation into memory cells. Induction of immune responses can be attempted by various methods and involves administration of antigens in different forms, with variable effect on the magnitude and quality of the immune response. One limiting factor in achieving a control of the immune response is targeting pAPC able to process and effectively present the resulting epitopes to specific T cells.

A solution to this problem is direct antigen delivery to secondary lymphoid organs, a microenvironment abundant in pAPC and T cells. The antigen can be delivered, for example, either as polypeptide or as an expressed antigen by any of a variety of vectors. The outcome in terms of magnitude and quality of immunity can be controlled by factors including, for example, the dosage, the formulation, the nature of the vector, and the molecular environment. Embodiments of the present invention can enhance control of the immune response. Control of the immune response includes the capability to induce different types of immune responses as needed, for example, from regulatory to pro-inflammatory responses. Preferred embodiments provide enhanced control of the magnitude and quality of responses to MHC class I-restricted epitopes which are of major interest for active immunotherapy.

Previous immunization methods displayed certain important limitations. First, very often, conclusions regarding the potency of vaccines were extrapolated from immunogenicity data generated from one or from a very limited panel of ultra sensitive read-out assays. Frequently, despite the inferred potency of a vaccination regimen, the clinical response was not significant or was at best modest. Secondly, subsequent to immunization, T regulatory cells, along with more conventional T effector cells, can be generated and/or expanded, and such cells can interfere with the function of the desired immune response. The importance of such mechanisms in active immunotherapy has been recognized only recently.

Intranodal administration of immunogens provides a basis for the control of the magnitude and profile of immune responses. The effective in vivo loading of pAPC accomplished as a result of such administration, enables a substantial magnitude of immunity, even by using an antigen in its most simple form—a peptide epitope—otherwise generally associated with poor pharmocokinetics. The quality of response can be further controlled via the nature of immunogens, vectors, and protocols of immunization. Such protocols can be applied for enhancing/modifying the response in tumoral processes.

Immunization has traditionally relied on repeated administration of antigen to augment the magnitude of the immune response. The use of DNA vaccines has resulted in high quality responses, but it has been difficult to obtain high magnitude responses using such vaccines, even with repeated booster doses. Both characteristics of the response, high quality and low magnitude, are likely due to the relatively low levels of epitope loading onto MHC achieved with these vectors. Instead it has become more common to boost such vaccines using antigen encoded in a live virus vector in order to achieve the high magnitude of response needed for clinical usefulness. However, the use of live vectors can entail several drawbacks including potential safety issues, decreasing effectiveness of later boosts due to a humoral response to the vector induced by the prior administrations, and the costs of creation and production. Thus, use of live vectors or DNA alone, although eliciting high quality responses, can result in a limited magnitude or sustainability of response.

Disclosed herein are embodiments that relate to protocols and to methods that, when applied to peptides, rendered them effective as immune therapeutic tools. Such methods circumvent the poor PK of peptides, and if applied in context of specific, and often more complex regimens, result in robust amplification and/or control of immune response. In preferred embodiments, direct administration of peptide into lymphoid organs results in unexpectedly strong amplification of immune responses, following a priming agent that induces a strong, moderate or even mild (at or below levels of detection by conventional techniques) immune response consisting of Tc1 cells. While preferred embodiments of the invention can employ intralymphatic or perilymphatic administration of antigen at all stages of immunization, intralymphatic administration is the most preferred mode of administration for adjuvant-free peptide. Peptide amplification utilizing intralymphatic administration can be applied to existing immune responses that may have been previously induced. Previous induction can occur by means of natural exposure to the antigen or by means of commonly used routes of administration, including without limitation subcutaneous, intradermal, intraperitoneal, intramuscular, and mucosal.

Also as shown herein, optimal initiation, resulting in subsequent expansion of specific T cells, can be better achieved by exposing the naïve T cells to limited amounts of antigen (as can result from the often limited expression of plasmid-encoded antigen) in a rich co-stimulatory context (such as in a lymph node). This can result in activation of T cells carrying T cell receptors that recognize, with high affinity, the MHC—peptide complexes on antigen presenting cells and can result in generation of memory cells that are more reactive to subsequent stimulation. The beneficial co-stimulatory environment can be augmented or ensured through the use of immunopotentiating agents and thus intralymphatic administration, while advantageous, is not in all embodiments required for initiation of the immune response. In embodiments involving the use of epitopic peptide for induction/entrainment it is preferred that a relatively low dosage of peptide (as compared to an amplifying dose or to a MHC-saturating concentration) be used so that presentation is limited, especially if using direct intralymphatic administration. Such embodiments generally involve inclusion of an immunopotentiator to achieve entrainment.

While the poor pharmacokinetics of free peptides has prevented their use in most routes of administration, direct administration into secondary lymphoid organs, particularly lymph nodes, has proven effective when the level of antigen is maintained more or less continuously by continuous infusion or frequent (for example, daily) injection. Such intranodal administration for the generation of CTL is taught in U.S. patent application Ser. Nos. 09/380,534, 09/776,232 (Pub. No. 20020007173 A1), now U.S. Pat. No. 6,977,074, and Ser. No. 11/313,152 (Pub. No. 20060153858), filed on Dec. 19, 2005), and in PCT Application No. PCTUS98/14289 (Pub. No. WO9902183A2), each entitled METHOD OF INDUCING A CTL RESPONSE, each of which is hereby incorporated by reference in its entirety. In some embodiments of the instant invention, intranodal administration of peptide was effective in amplifying a response initially induced with a plasmid DNA vaccine. Moreover, the cytokine profile was distinct, with plasmid DNA induction/peptide amplification generally resulting in greater chemokine (chemoattractant cytokine) and lesser immunosuppressive cytokine production than either DNA/DNA or peptide/peptide protocols.

Thus, such DNA induction/peptide amplification protocols can improve the effectiveness of compositions, including therapeutic vaccines for cancer and chronic infections. Beneficial epitope selection principles for such immunotherapeutics are disclosed in U.S. patent application Ser. Nos. 09/560,465, 10/026,066 (Pub. No. 20030215425 A1), Ser. No. 10/005,905, filed Nov. 7, 2001, Ser. No. 10/895,523 (Pub. No. 20050130920 A1), filed Jul. 20, 2004, and Ser. No. 10/896,325 (Pub No. ______), filed Jul. 20, 2004, all entitled EPITOPE SYNCHRONIZATION IN ANTIGEN PRESENTING CELLS; Ser. No. 09/561,074, now U.S. Pat. No. 6,861,234, and Ser. No. 10/956,401 (Pub. No. 20050069982 A1), filed on Oct. 1, 2004, both entitled METHOD OF EPITOPE DISCOVERY; Ser. No. 09/561,571, filed Apr. 28, 2000, entitled EPITOPE CLUSTERS; Ser. No. 10/094,699 (Pub. No. 20030046714 A1), filed Mar. 7, 2002, Ser. No. 11/073,347, (Pub. No. 20050260234), filed Jun. 30, 2005, each entitled ANTI-NEOVASCULATURE PREPARATIONS FOR CANCER; and Ser. No. 10/117,937 (Pub. No. 20030220239 A1), filed Apr. 4, 2002, Ser. No. 11/067,159 (Pub. No. 20050221440A1), filed Feb. 25, 2005, Ser. No. 10/067,064 (Pub. No. 20050142114 A1), filed Feb. 25, 2005, and Ser. No. 10/657,022 (Publication No. 20040180354 A1), and PCT Application No. PCT/US2003/027706 (Pub. No. WO 04/022709 A2), each entitled EPITOPE SEQUENCES, and each of which is hereby incorporated by reference in its entirety. Aspects of the overall design of vaccine plasmids are disclosed in U.S. patent application Ser. No. 09/561,572, filed Apr. 28, 2000, and Ser. No. 10/225,568 (Pub. No. 20030138808 A1), filed Aug. 20, 2002, both entitled EXPRESSION VECTORS ENCODING EPITOPES OF TARGET-ASSOCIATED ANTIGENS and U.S. patent application Ser. No. 10/292,413 (Pub. No. 20030228634 A1), Ser. No. 10/777,053 (Pub. No. 20040132088 A1), filed on Feb. 10, 2004, and Ser. No. 10/837,217 (Pub. No. 20040203051), filed on Apr. 30, 2004, all entitled EXPRESSION VECTORS ENCODING EPITOPES OF TARGET-ASSOCIATED ANTIGENS AND METHODS FOR THEIR DESIGN; Ser. No. 10/225,568 (Pub No. 20030138808 A1), PCT Application No. PCT/US2003/026231 (Pub. No. WO 2004/018666) and U.S. Pat. No. 6,709,844 and U.S. patent application Ser. No. 10/437,830 (Pub. No. 20030180949 A1), filed on May 13, 2003, each entitled AVOIDANCE OF UNDESIRABLE REPLICATION INTERMEDIATES IN PLASMID PROPAGATION, each of which is hereby incorporated by reference in its entirety. Specific antigenic combinations of particular benefit in directing an immune response against particular cancers are disclosed in provisional U.S. Provisional Application No. 60/479,554, filed on Jun. 17, 2003, U.S. patent application Ser. No. 10/871,708 (Pub. No. 20050118186 A1), filed on Jun. 17, 2004, PCT Patent Application No. PCT/US2004/019571 (Pub. No. WO 2004/112825), U.S. Provisional Application No. 60/640,598, filed Dec. 29, 2005, and U.S. patent application Ser. No. 11/323,049 (Pub. No. 20060159694), filed on Dec. 29, 2005, all entitled COMBINATIONS OF TUMOR-ASSOCIATED ANTIGENS IN VACCINES FOR VARIOUS TYPES OF CANCERS, each of which is also hereby incorporated by reference in its entirety. The use and advantages of intralymphatic administration of BRMs are disclosed in provisional U.S. Patent Application No. 60/640,727, filed Dec. 29, 2005 and U.S. patent application Ser. No. 11/321,967(Pub. No. 20060153844), filed on Dec. 29, 2005, both entitled Methods to trigger, maintain and manipulate immune responses by targeted administration of biological response modifiers into lymphoid organs, each of which is incorporated herein by reference in it entirety. Additional methodology, compositions, peptides, and peptide analogues are disclosed in U.S. patent application Ser. No. 09/999,186, filed Nov. 7, 2001, entitled METHODS OF COMMERCIALIZING AN ANTIGEN; and U.S. Provisional U.S. Patent Application No. 60/640,821, filed Dec. 29, 2005 and application Ser. No. 11/323,520 (Pub. No. ______), filed on Dec. 29, 2005, both entitled METHODS TO BYPASS CD4+ CELLS IN THE INDUCTION OF AN IMMUNE RESPONSE, each of which is hereby incorporated by reference in its entirety.

Other relevant disclosures are present in U.S. patent application Ser. No. 11/156,369 (Pub. No. 20060057673), and U.S. Provisional Patent Application No. 60/691,889, both filed on Jun. 17, 2005, both entitled EPITOPE ANALOGS, and each of which is incorporated herein by reference in its entirety. Also relevant are, U.S. Provisional Patent App. No. 60/691,579, filed on Jun. 17, 2005, entitled METHODS AND COMPOSITIONS TO ELICIT MULTIVALENT IMMUNE RESPONSES AGAINST DOMINANT AND SUBDOMINANT EPITOPES, EXPRESSED ON CANCER CELLS AND TUMOR STROMA, and 60/691,581, filed on Jun. 17, 2005, entitled MULTIVALENT ENTRAIN-AND-AMPLIFY IMMUNOTHERAPEUTICS FOR CARCINOMA, each of which is incorporated herein by reference in its entirety.

Protocols involving specific sequences of recombinant DNA entrainment doses, followed by peptide boosts administered to lymphoid organs, are useful for the purpose of induction, amplification and maintenance of strong T cell responses, for example, for prophylaxis or therapy of infectious or neoplastic diseases. Such diseases can be carcinomas (e.g., renal, ovarian, breast, lung, colorectal, prostate, head-and-neck, bladder, uterine, skin), melanoma, tumors of various origin and in general tumors that express defined or definable tumor associated antigens, such as oncofetal (e.g., CEA, CA 19-9, CA 125, CRD-BP, Das-1, 5T4, TAG-72, and the like), tissue differentiation (e.g., Melan-A, tyrosinase, gp100, PSA, PSMA, and the like), or cancer-testis antigens (e.g., PRAME, MAGE, LAGE, SSX2, NY-ESO-1, and the like). Cancer-testis genes and their relevance for cancer treatment are reviewed in Scanlon et al., (see Cancer Immunity 4:1-15, 2004, which is hereby incorporated by reference in its entirety). Antigens associated with tumor neovasculature (e.g., PSMA, VEGFR2, Tie-2) are also useful in connection with cancerous diseases, as is disclosed in U.S. patent application Ser. No. 10/094,699 (Pub. No. 20030046714 A1) and Ser. No. 11/073,347 (Pub. No. 20050260234), filed on Jun. 30, 2005, entitled ANTI-NEOVASCULATURE PREPARATIONS FOR CANCER, each of which is hereby incorporated by reference in its entirety.

Preferred applications of entrain and amplify methods include injection or infusion into one or more lymph nodes, starting with a number (e.g., 1 to 10, or more, 2 to 8, 3 to 6, preferably about 4 or 5) of administrations of recombinant DNA (dose range of 0.001-10 mg/kg, preferably 0.005-5 mg/kg) followed by one or more (preferably about 2) administrations of peptide, preferably in an immunologically inert vehicle or formulation (dose range of 1 ng/kg-10 mg/kg, preferably 0.005-5 mg/kg). Because dose does not necessarily scale linearly with the size of the subject, doses for humans can tend toward the lower, and doses for mice can tend toward the higher, portions of these ranges. The preferred concentration of plasmid and peptide upon injection is generally about 0.1 μg/ml-10 mg/ml, and the most preferred concentration is about 1mg/ml, generally irrespective of the size or species of the subject. However, particularly potent peptides can have optimum concentrations toward the low end of this range, for example between 1 and 100 μg/ml. When peptide only protocols are used to promote tolerance, doses toward the higher end of these ranges are generally preferred (e.g., 0.5-10 mg/ml). This sequence can be repeated as long as necessary to maintain a strong immune response in vivo. Moreover, the time between the last entraining dose of DNA and the first amplifying dose of peptide is not critical. Preferably it is about 7 days or more, and can exceed several months. The multiplicity of injections of the DNA and/or the peptide can be reduced by substituting infusions lasting several days (preferably 2-7 days). It can be advantageous to initiate the infusion with a bolus of material similar to what might be given as an injection, followed by a slow infusion (24-12000 μl/day to deliver about 25-2500 μg/day for DNA, 0.1-10,000 μg/day for peptide). This can be accomplished manually or through the use of a programmable pump, such as an insulin pump. Such pumps are known in the art and enable periodic spikes and other dosage profiles, which can be desirable in some embodiments.

In preferred embodiments the method calls for direct administration to the lymphatic system. In preferred embodiments this is to a lymph node. Afferent lymph vessels are similarly preferred. Choice of lymph node is not critical. Inguinal lymph nodes are preferred for their size and accessibility, but axillary and cervical nodes and tonsils can be similarly advantageous. Administration to a single lymph node can be sufficient to induce or amplify an immune response. Administration to multiple nodes can increase the reliability and magnitude of the response. For embodiments promoting a multivalent response and in which multiple amplifying peptides are therefore used, it can be preferable that only a single peptide be administered to any particular lymph node on any particular occasion. Thus, one peptide can be administered to the right inguinal lymph node and a second peptide to the left inguinal lymph node at the same time, for example. Additional peptides can be administered to other lymph nodes even if they were not sites of induction, as it is not essential that initiating and amplifying doses be administered to the same site, due to migration of T lymphocytes. Alternatively any additional peptides can be administered a few days later, for example, to the same lymph nodes used for the previously administered amplifying peptides since the time interval between induction and amplification generally is not a crucial parameter, although in preferred embodiments the time interval can be greater than about a week. Segregation of administration of amplifying peptides is generally of less importance if their MHC-binding affinities are similar, but can grow in importance as the affinities become more disparate. Incompatible formulations of various peptides can also make segregated administration preferable.

Patients that can benefit from such methods of immunization can be recruited using methods to define their MHC protein expression profile and general level of immune responsiveness. In addition, their level of immunity can be monitored using standard techniques in conjunction with access to peripheral blood. Finally, treatment protocols can be adjusted based on the responsiveness to induction or amplification phases and variation in antigen expression. For example, repeated entrainment doses preferably can be administered until a detectable response is obtained, and then administering the amplifying peptide dose(s), rather than amplifying after some set number of entrainment doses. Similarly, scheduled amplifying or maintenance doses of peptide can be discontinued if their effectiveness wanes, antigen-specific regulatory T cell numbers rise, or some other evidence of tolerization is observed, and further entrainment can be administered before resuming amplification with the peptide. The integration of diagnostic techniques to assess and monitor immune responsiveness with methods of immunization is discussed more fully in Provisional U.S. Patent Application No. 60/580,964, which was filed on Jun. 17, 2004 and U.S. patent application Ser. No. 11/155,928 (Pub. No. 20050287068), filed Jun. 17, 2005, both entitled IMPROVED EFFICACY OF ACTIVE IMMUNOTHERAPY BY INTEGRATING DIAGNOSTIC WITH THERAPEUTIC METHODS, each of which is hereby incorporated by reference in its entirety.

Practice of many of the methodological embodiments of the invention involves use of at least two different compositions and at least one chemotherapeutic agent. In embodiments where there is more than a single target antigen, the methods can involve several immunogenic composition(s) and chemotherapeutic agent(s) to be administered together and/or at different times. Thus, embodiments of the invention include sets and subsets of chemotherapeutic agent(s) and immunogenic composition(s) and individual doses thereof. Multivalency can be achieved using compositions comprising multivalent immunogens, combinations of monovalent immunogens, coordinated use of compositions comprising one or more monovalent immunogens or various combinations thereof. Multiple compositions, manufactured for use in a particular treatment regimen or protocol according to such methods, define an immunotherapeutic product. In some embodiments all or a subset of the compositions of the product are packaged together in a kit along with or separate from the chemotherapeutic agent(s). In some instances the inducing and amplifying compositions targeting a single epitope, or set of epitopes, can be packaged together. In other instances multiple inducing compositions can be assembled in one kit and the corresponding amplifying compositions assembled in another kit. Alternatively compositions can be packaged and sold individually along with instructions, in printed form or on machine-readable media, describing how they can be used in conjunction with each other to achieve the beneficial results of the methods of the invention. Further variations will be apparent to one of skill in the art. The use of various packaging schemes comprising less than all of the agents and/or compositions that might be employed in a particular protocol or regimen facilitates the personalization of the treatment, for example based on tumor antigen expression, or observed response to the immunotherapeutic or its various components, as described in U.S. Provisional Application No. 60/580,969, filed on Jun. 17, 2004, U.S. patent application Ser. No. 11/155,288 (Pub. No. 20060008468) filed Jun. 17, 2005, and U.S. patent application Ser. No. 11/323,964, filed Dec. 29, 2005, all entitled COMBINATIONS OF TUMOR-ASSOCIATED ANTIGENS IN DIAGNOSTICS FOR VARIOUS TYPES OF CANCERS; and Provisional U.S. Patent Application No. 60/580,964, and U.S. patent application Ser. No. 11/155,928 (Pub. No. 20050287068, both entitled IMPROVED EFFICACY OF ACTIVE IMMUNOTHERAPY BY INTEGRATING DIAGNOSTIC WITH THERAPEUTIC METHODS, each of which is incorporated by reference in its entirety above. COMBINATION THERAPIES AND DELIVERY

In particular embodiments of the invention there is provided a therapeutic approach comprising an immunotherapeutic regimen in combination with a chemotherapeutic agent that depletes T-regulatory cells thereby enabling T cell activity within a tumor. Preferably, the chemotherapeutic agent is cyclophosphamide.

In combination with the immunotherapeutic/chemotherapeutic strategies disclosed herein, other therapeutic strategies can also be employed. Other cancer therapies contemplated include, in a non-limiting manner, radiotherapy, biotherapy, gene therapy, hormonal therapy, or surgery.

Other therapies that can be employed in combination with the immunotherapeutic/chemotherapeutic strategy described herein include, but are not limited to: immune adjuvants (e.g., Mycobacterium bovis, Plasmodium falciparum, dinitrochlorobenzene and aromatic compounds); cytokine therapy (e.g., interferons alpha, beta and gamma; IL-1, GM-CSF and TNF); and monoclonal antibodies (e.g., anti-ganglioside GM2, anti-HER-2, anti-p185).

Other chemotherapeutic agents well known to those of ordinary skill in the art, can also be employed in the methods and combination strategies disclosed herein. These include, in a non-limiting manner, for example, gemcitabine, fludarabine, cisplatin (CDDP), carboplatin, procarbazine, mechlorethamine, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristine, vinblastine and methotrexate or any analog or derivative variant thereof.

In still other embodiments, surgery, such as curative surgery can be employed in combination with the immunotherapeutic/chemotherapeutic strategy disclosed herein. Curative surgery for cancer includes resection in which all or part of cancerous tissue is physically removed, excised, and/or destroyed.

Various parameters can be taken into account in delivering or administering an immunotherapeutic and/or chemotherapeutic composition to a subject. In addition, a dosage regimen and immunization schedule can be employed. Generally the amount of the components in the therapeutic composition will vary from patient to patient and from antigen to antigen, depending on such factors as: the activity of the antigen in inducing a response; the flow rate of the lymph through the patient's system; the weight and age of the subject; the type of disease and/or condition being treated; the severity of the disease or condition; previous or concurrent therapeutic interventions; the capacity of the individual's immune system to synthesize antibodies; the degree of protection desired; the manner of administration and the like, all of which can be readily determined by the practitioner.

In general the therapeutic composition can be delivered at a rate of from about 1 to about 500 microliters/hour or about 24 to about 12000 microliters/day. The concentration of the antigen is such that about 0.1 micrograms to about 10,000 micrograms of the antigen will be delivered during 24 hours. The flow rate is based on the knowledge that each minute approximately about 100 to about 1000 microliters of lymph fluid flows through an adult inguinal lymph node. The objective is to maximize local concentration of vaccine formulation in the lymph system. A certain amount of empirical investigation on patients will be necessary to determine the most efficacious level of infusion for a given vaccine preparation in humans.

The immunotherapeutic and/or chemotherapeutic compositions can include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. Also of importance is the subject to be treated, in particular, the state of the subject and the protection desired. A unit dose need not be administered as a single injection but can comprise continuous infusion over a set period of time.

In particular embodiments, the immunotherapeutic and/or chemotherapeutic composition can be administered as a plurality of sequential doses. Such plurality of doses can be 2, 3, 4, 5, 6 or more doses as is needed. In further embodiments of the present invention, it is contemplated that the doses of the immunotherapeutic and/or chemotherapeutic composition can be administered within about seconds or minutes of each other into the right or left inguinal lymph nodes. For example, the plasmid (prime) can first be injected into the right lymph node followed within seconds or minutes by a second plasmid into the left inguinal lymph node. In other instances the combination of one or more plasmids expressing one or more immunogens can be administered. It is preferred that the subsequent injection following the first injection into the lymph node be within at about 1, 2, 3, 4, 5, 6, 7, 8, 9, 10 or more minutes but not greater than about 30, 40, 50, or 60 minutes of the first injection. Similar considerations apply to the administration of two peptides individually to the right and left lymph nodes. It can be desirable to administer the plurality of doses of the immunotherapeutic and/or chemotherapeutic composition of the invention at an interval of days, where several days (1, 2, 3, 4, 5, 6, or 7, or more days) lapse between subsequent administrations. In other instances it can be desirable for subsequent administration(s) of the therapeutic compositions of the invention to be administered via bilateral inguinal lymph node injection within about 1, 2, 3, or more weeks or within about 1, 2, 3, or more months following the initial dose administration.

Administration can be in any manner compatible with the dosage formulation and in such amount as will be therapeutically effective. An effective amount or dose of an immunotherapeutic and/or chemotherapeutic composition of the present invention is that amount needed to provide a desired response in the subject to be treated. An effective amount is described, generally, as that amount sufficient to detectably and repeatedly to ameliorate, reduce, minimize or limit the extent of the disease or its symptoms. More rigorous definitions can apply, including elimination, eradication or cure of disease.

Preferably, immunomodulatory doses (usually low doses) of chemotherapy designed to selectively deplete T-regulatory cells to enhance immune responsiveness prior to immunotherapy can be provided according to currently approved medical standards taking into account the toxicity.

In some embodiments, the numbers expressing quantities of ingredients, properties such as molecular weight, reaction conditions, and so forth used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention can contain certain errors necessarily resulting from the standard deviation found in their respective testing measurements.

In some embodiments, the terms “a” and “an” and “the” and similar referents used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in its entirety.

It is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, the present invention is not limited to that precisely as shown and described.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Tumor Regression Elicited by Targeted Lymph Node Immunotherapy with an HPV-16 (E7) Peptide

Tumor regression elicited by targeted lymph node immunotherapy was assessed in an HPV-16 tumor model, by in vivo loading of lymph node APCs with the E7₄₉₋₅₇ peptide in combination with an adjuvant acting via TLRs (synthetic dsRNA), to elicit a potent MHC class I-restricted immunity.

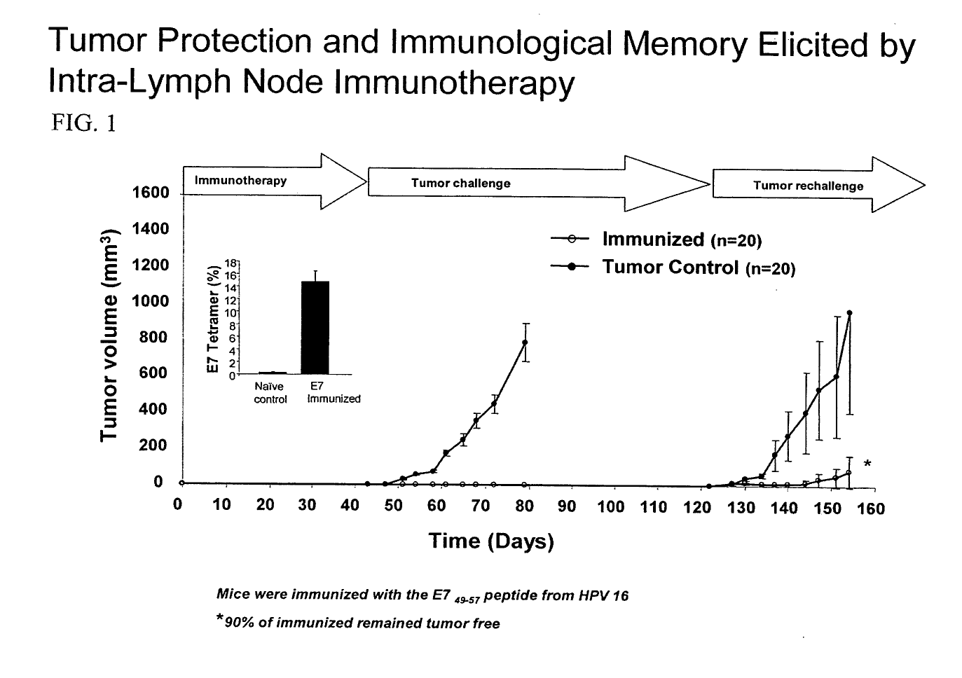

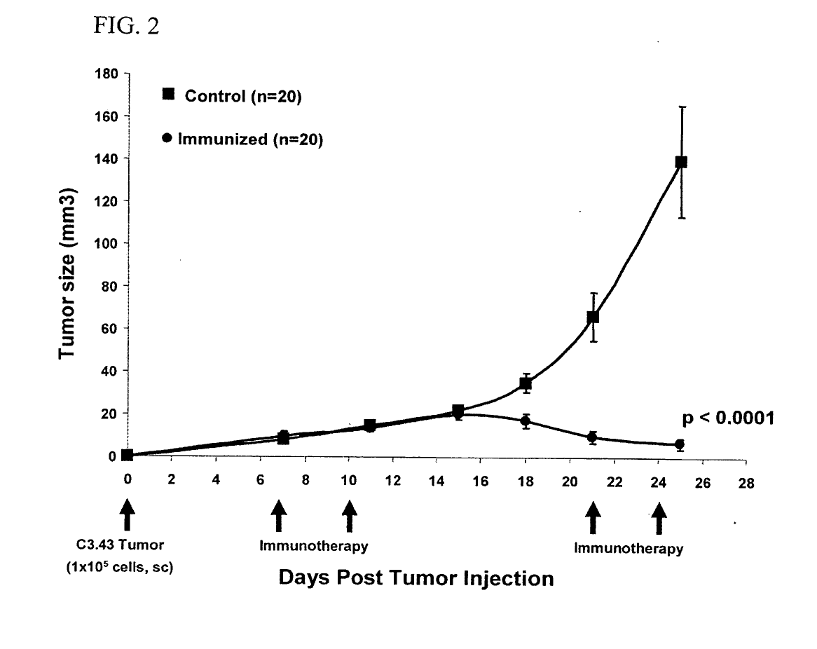

Mice bearing human papillomavirus type 16-transformed tumors received intranodal injections of a MHC class I HPV-16 E7₄₉₋₅₇ peptide co-injected with double stranded RNA (polyIC) as an adjuvant on day seven following subcutaneous tumor (10⁵ cells) challenge (FIG. 1). The majority of immunized mice (60%) were completely cured with 7 out of 20 showing complete protection (CP) and 5 out of 20 forming a measurable tumor which completely responded (CR) following immunotherapy on Days 7, 10, 21, and 24 (FIG. 2; Table 1). One animal demonstrated a partial response (PR) resulting in a tumor that was 32% smaller at the end of the treatment regimen (Table 1). Caliper measurements and ultrasound imaging techniques were used to monitor tumor progression and assess tumor free survival.

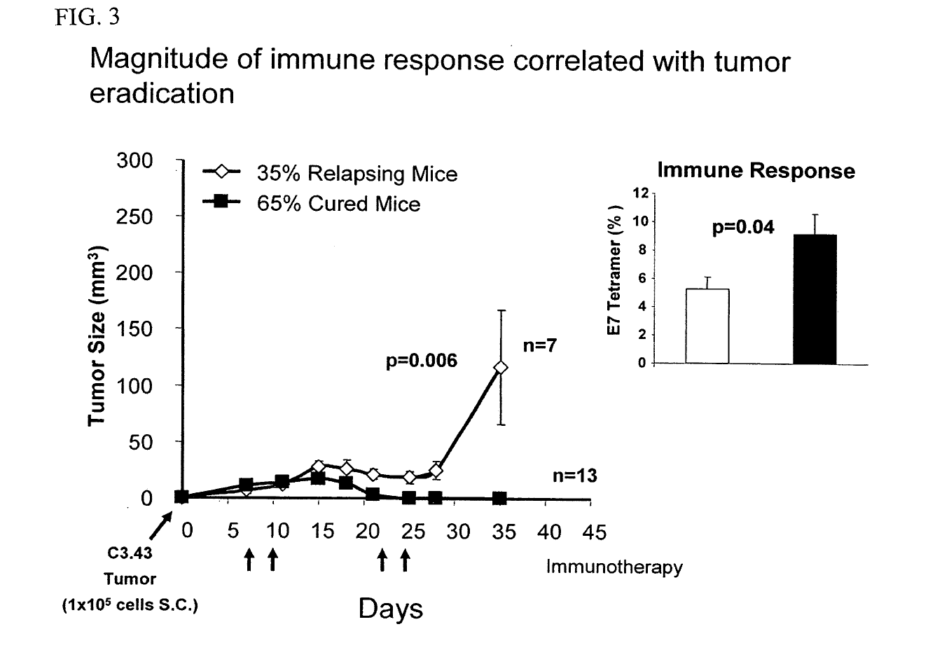

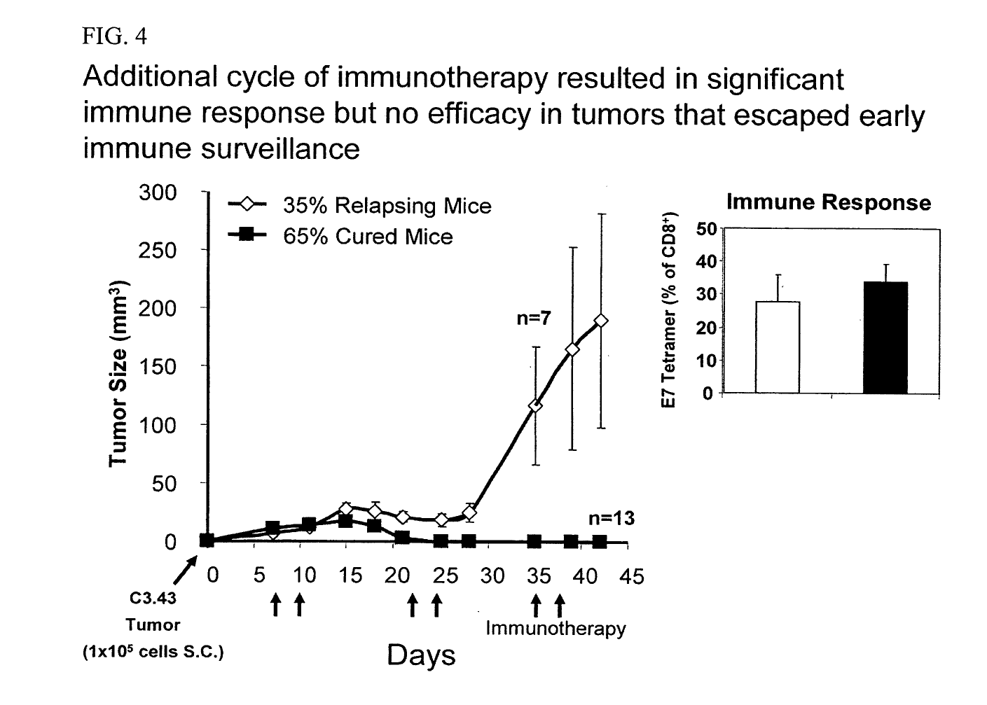

Tumor progression (PD) in the remaining animals was significantly delayed (FIG. 3) and correlated with lower initial antigen specific CTL responses, as shown by tetramer analysis. An additional round (boost) of immunotherapy on Days 35 and 38 significantly increased the immune response in progressing mice from an average of 5 to 30%, as shown by tetramer analysis (FIGS. 3 and 4 right panels) however; no improvement on tumor efficacy was observed (FIGS. 3 and 4).

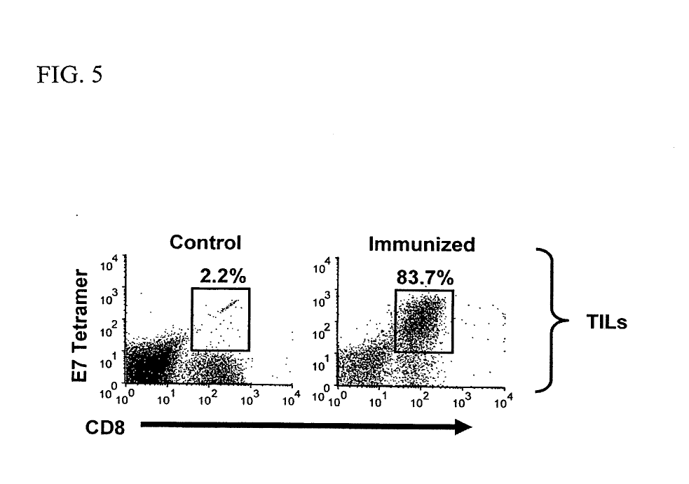

As shown in FIG. 5, isolation of TILs from tumors confirmed the presence of HPV specific CD8⁺ cells (83.7%) in the immunized mice population as compared to that of the control (2.2%). This data indicated that the function of TILs was impaired and the lack of improvement on tumor efficacy can be due to other factors such as, for example, the tumor micro-environment.

TABLE 1
Protection from HPV type 16- transformed tumors in mice

| | | Response | Tumor | Initiation | Final Tumor |
|---|---|---|---|---|---|
| | Disease | Rate (%), | Variance | Tumor Size, | Size, Day |
| Treatment | Class | n/20 mice | (%) | Day 7 (mm³) | 32 (mm³) |
| Immuno- | ¹CP | 35, 7/20 | 0 | 0 | 0 |
| therapy | ²CR | 25, 5/20 | −100 | 25 | 0 |
| on day 7 | ³PR | 5, 1/20 | −32 | 30 | 20 |
| | ⁴PD | 35, 7/20 | 1181 | 20 | 194 |

¹Complete Protection,
²Complete Response,
³Partial Response,
⁴Progressive Disease

### Example 2

### Increased Frequency of T-Regulatory Cells in Progressive Disease

To assess the role of T-regulatory cells in animals that failed to respond to immunotherapy, mice bearing human papillomavirus type 16-transformed tumors received intranodal injections of a MHC class I HPV-16 E7₄₉₋₅₇ peptide co-injected with double stranded RNA (polyIC) as an adjuvant on days 21, 25, 35, and 39 following subcutaneous tumor (10⁵ cells) challenge. Control mice received either poyIC or saline.

Additionally, to determine the potential tolerance of HPV-specific tumor infiltrating lymphocytes (TILs) to the immuno-modulatory effects of cyclophosphamide, cyclophosphamide was employed in combination with the HPV-16 E7₄₉₋₅₇ peptide immunotherapeutic strategy disclosed in Example 1. Cyclophosphamide is an alkylating chemotherapeutic agent that has been shown to have cytotoxic as well as immuno-modulatory effects, such as depletion of CD4⁺CD25⁺ regulatory T cells and enhancement of antigen specific CTL responses which have resulted in increased tumor efficacy (Ercolini A M, et al., J Exp Med., 16;201(10):1591-602, 2005; Lutsiak M E, et al., Blood. April 1;105(7):2862-8, 2005; Hermans I F, et al., Cancer Research 63, 8408-8413, 2003; Loeffler M, et al., Cancer Res, 65:12, 2005).

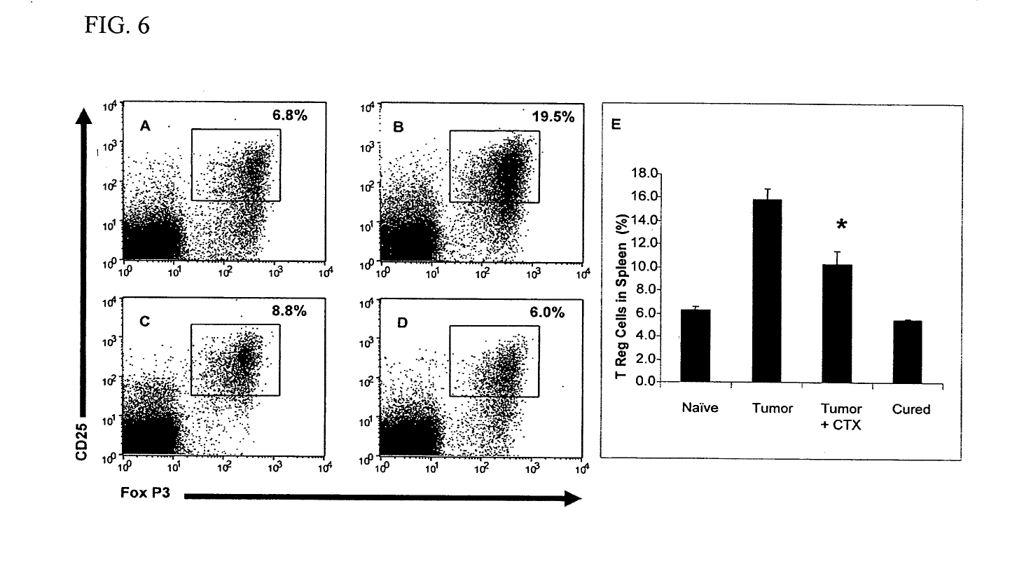

Mice received one injection of cyclophosphamide (CTX, 100 mg/kg) on days 46 and 50. On day 49, spleens were removed from 3 mice in each group and the percentage of CD25⁺ and Fox P3⁺ cells were calculated within the total CD4⁺ population (FIG. 6). The data shows that mice with progressing tumors (Panel B) had approximately 3 fold more T-regulatory cells compared to the naïve (Panels A and E) or cured (Panel D) mice. Mice with tumors that received one injection of cyclophosphamide (CTX, 100 mg/kg) on Day 46 had significantly reduced levels of (Students T test, p value=0.02) T-regulatory cells (FIG. 6, Panels C and E).

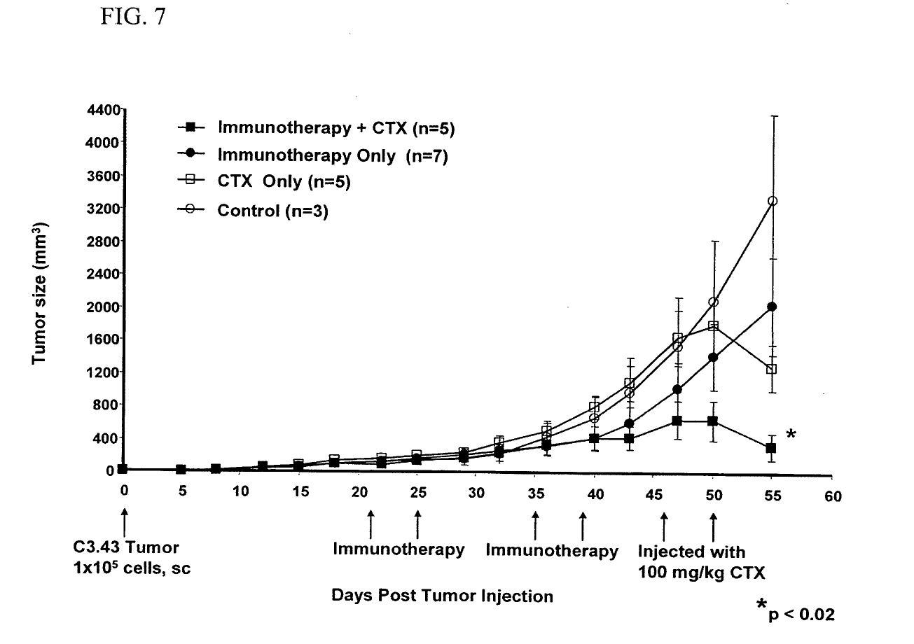

In addition, combination therapy employing both the HPV-16 E7₄₉₋₅₇ peptide immunotherapeutic strategy with cyclophosphamide (FIG. 7) resulted in antitumor activity that was dramatically enhanced (p<0.02) over either treatment administered alone. These results indicate that combinatorial therapeutic approaches (as disclosed elsewhere herein) potentiate the efficacy of active cancer immunotherapy. These findings provide a new rationale for the combination of chemotherapy and immunotherapy in cancer treatment.

### Example 3

### Administration of a Chemotherapeutic Agent Prior to the Immunotherapeutic Regimen

Additional studies are conducted wherein non-limiting chemotherapeutic agents such as, for example, but not limited to, cyclophosphamide, gemcitabine, fludarabine and doxorubicin are employed to selectively deplete T-regulatory cells to enhance immune responsiveness prior to immunotherapy. Using a similar strategy as disclosed in Example 1 above, mice bearing human papillomavirus type 16-transformed tumors first received immunomodulatory doses (low doses) of a chemotherapeutic agent followed at various intervals by intranodal injections of a MHC class I HPV-16 E7₄₉₋₅₇ peptide co-injected with double stranded RNA (polyIC) as an adjuvant. Mice are then assessed for regression of tumor.

Dosing is according to currently approved medical standards as are known to one of ordinary skill in the art. The therapeutic regimen, chemotherapy followed by lymph node targeted immunotherapy, is optionally repeated several times to improve tumor efficacy.

### Example 4

### Plasmid Priming Combined with Peptide Boosting Strategy

In order to assess whether plasmid priming combined with peptide boosting strategy produces results similar to those observed in Examples 1-3 above, a chemotherapeutic is administered for one week followed by plasmid priming (pROC, pBPL, pSEM as disclosed elsewhere herein) on various days, for example, on Days 8, 11, 22, and 25. The immune response is then boosted with peptide (PRAME₄₂₅₋₄₃₃, PSMA₂₈₈₋₂₉₇, NY-ESO 1₁₅₇₋₁₆₅, SSX-2₄₁₋₄₉, Melan-A₂₆₋₃₅, Tyrosinase₃₆₉₋₃₇₇ and analogues thereof, as disclosed elsewhere herein) on Days 36 and 40, for example. One week after the first therapeutic cycle, a second therapeutic cycle is optionally repeated.

### Example 5

### Ex vivo Peptide Loading of DCs Strategy

In order to assess whether the ex vivo peptide loading of DCs strategy produces results similar to those observed in Examples 1-3 above, peripheral blood is isolated from subjects for the culture of DCs, prior to chemotherapy. A chemotherapeutic agent is administered and then DCs loaded with peptide (PRAME₄₂₅₋₄₃₃, PSMA₂₈₈₋₂₉₇, NY-ESO-1₁₅₇₋₁₆₅, SSX-2₄₁₋₄₉, Melan A₂₆₋₃₅, Tyrosinase₃₆₉₋₃₇₇ and analogues thereof) are injected into the lymph node. One week after the first procedure, a second procedure can be repeated.

### Example 6

### Administration Using Single Versus Multiple Antigens Approach in Combination with Chemotherapy

In other studies, immunomodulatory metronomic doses of chemotherapy are provided throughout the immunotherapy therapeutic cycle to assess the effect on tumor regression. In this study, a chemotherapeutic agent is administered the first day of each week throughout the immunization cycle with plasmid priming (pROC, pBPL, pSEM) occurring on Days 8, 11, 22, and 25, for example, and peptide boosting (PRAME₄₂₅₋₄₃₃, PSMA₂₈₈₋₂₉₇, NY-ESO-1₁₅₇₋₁₆₅, SSX-2₄₁₋₄₉, Melan A₂₆₋₃₅, Tyrosinase₃₆₉₋₃₇₇ and analogues thereof) on Days 36 and 40, for example. One week after the first therapeutic cycle, a second therapeutic cycle can be repeated.

Studies are further conducted to assess the tumor efficacy when the chemotherapeutic agent is provided after plasmid (prime)/peptide (boost). This strategy is advantageous in the case of bulky or metastatic diseases (tumors) in that the subject is immunized first with plasmid on Days 1, 4, 15, 18 and boosted with peptide on Days 29 and 32. Immunotherapy is followed with chemotherapy after one week of rest to deplete T-regulatory cells, resulting in a reduction of T cell tolerance and unleashing of the effector potential of the tumor specific CTL in the tumor microenvironment.

### Example 7

### Protection from Disseminated Disease Following Intravenous HPV-16 Tumor Challenge

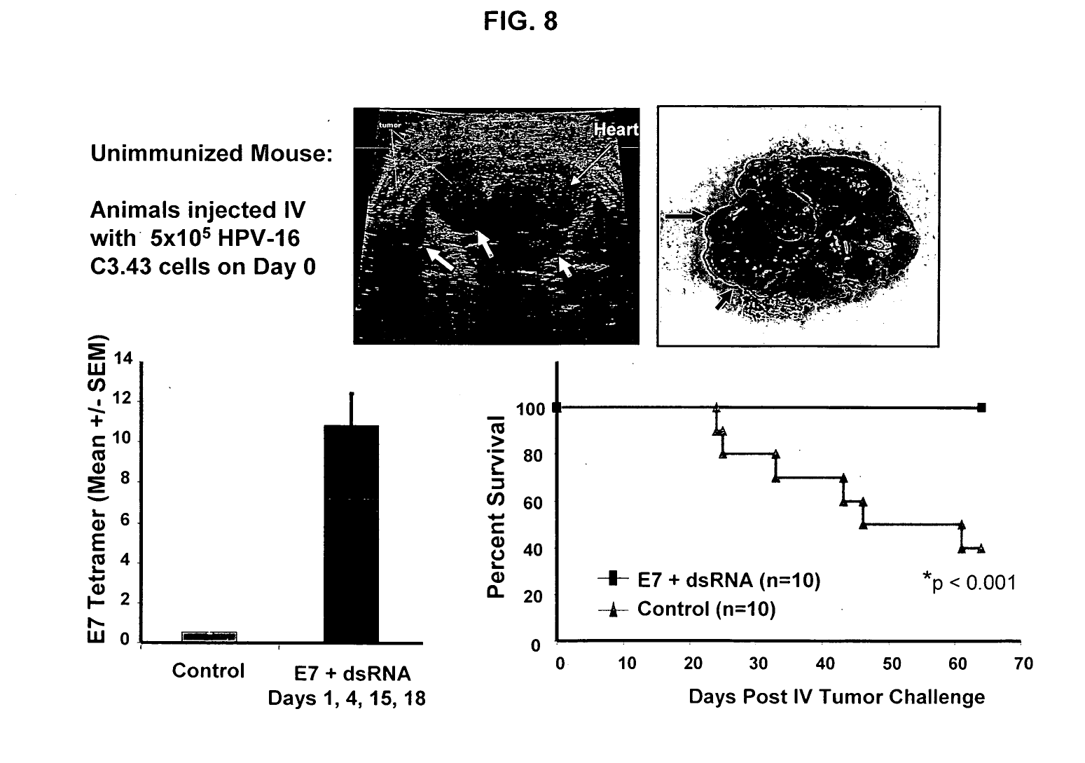

To evaluate immunological protection from disseminated disease, C57BL/6 mice (n=10) were injected intravenously with 5×10⁵ HPV-16 transformed tumor cells (C3.43) and then immunized in the bilateral inguinal lymph nodes with 12.5 μg E749-57 HPV peptide and 12.5 μg dsRNA (polyIC) as adjuvant per node on days 1, 4, 15, and 18 post tumor challenge. The immune response was measured by E749-57 Tetramer on Day 25 from peripheral blood (FIG. 8, Panel A) and percent survival for each group was calculated (FIG. 8, Panel B) and compared to untreated tumor challenged control mice (n=10). Immunized mice generated significant HPV-16 specific immune responses with an average of 10.5% and were completely protected from IV challenge of HPV-16 tumor cells out to Day 65. As expected, untreated mice displayed background levels of E7 Tetramer staining with only 40% of animals alive at Day 65. Death in the untreated animals was found to be due to tumor micro-metastases in the lungs as confirmed by ultrasound and necropsy postmortem.

### Example 8

### Targeted Lymph Node Administration of Antigen Significantly Improves Anti-Tumor Efficacy of HPV Cancer Immunotherapy

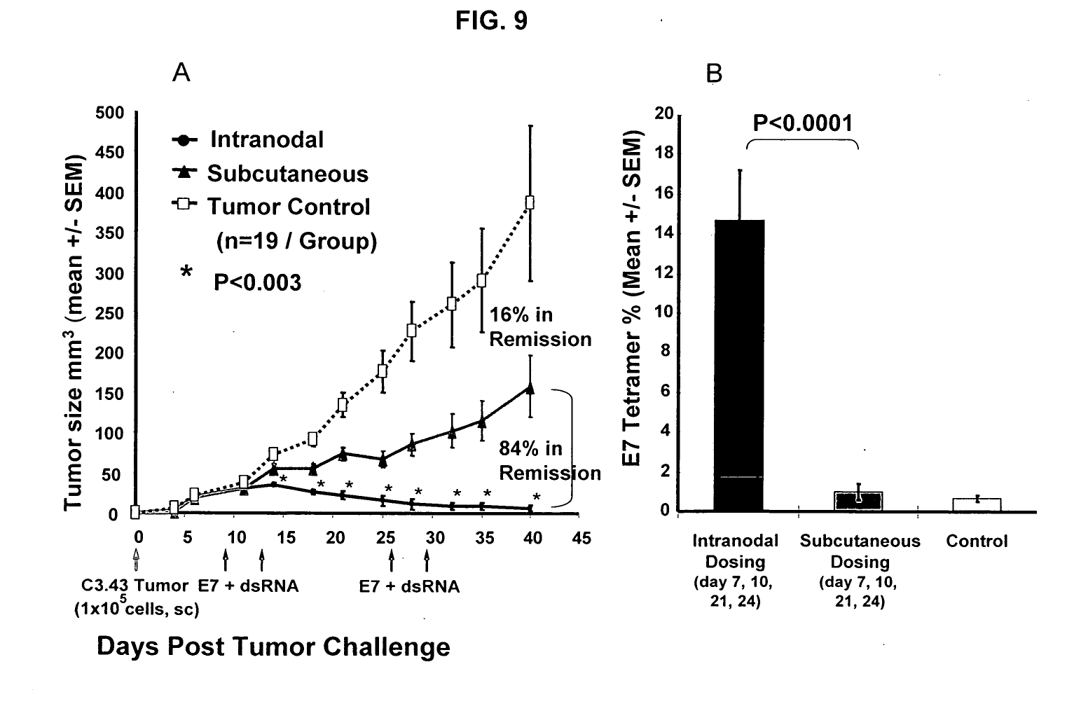

In a therapeutic model of HPV-16, the anti-tumor efficacy of intranodal versus conventional dosing was compared. C57BL/6 mice were subcutaneously challenged with 10⁵ HPV tumor cells on Day 0 and then immunized with 2.5 μg E7₄₉₋₅₇ HPV antigen and 25 μg dsRNA (polyIC) in bilateral inguinal lymph nodes (n=1 9) or subcutaneously (n=19) on Days 7, 10, 21, and 24. The immune response was measured by E7₄₉₋₅₇ Tetramer staining on Day 31 from peripheral blood (FIG. 9, Panel B) and tumor size for each group was calculated (FIG. 9, Panel A) and compared to untreated tumor challenged control mice (n=19). Lymph node immunized mice generated statistically significant HPV-16 specific immune responses with an average of 14.5% compared to subcutaneously dosed mice (p<0.0001). In addition, tumors in mice immunized in the lymph node began to regress on Day 15 resulting in 84% of animals in remission at Day 40. This response was significantly superior to animals dosed subcutaneously (p<0.003) whose tumor progression was only delayed compared to tumor controls with only 16% of animals resulting in disease remission. Untreated tumor control mice displayed background levels of E7 Tetramer staining (Panel B) and their tumors progressed exponentially without regression as expected (Panel A).

### Example 9

### Mice with Refractory/Progressing Tumors Showed Increased Levels of CD4⁺/CD25⁺/FoxP3^{HI} T-Regulatory Cells

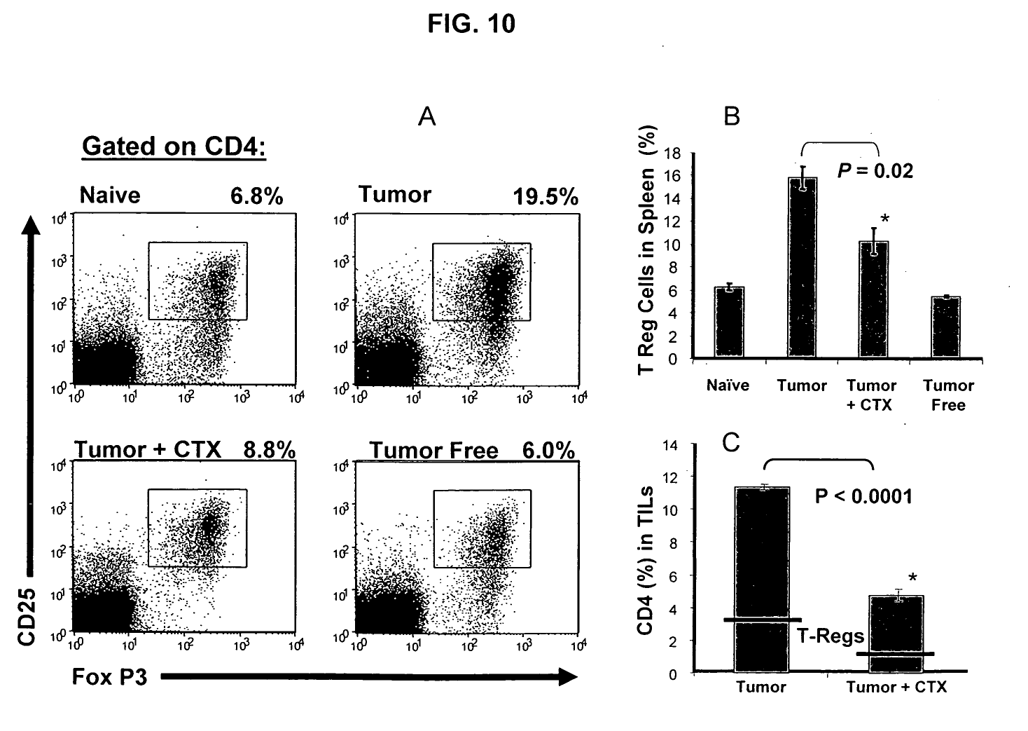

C57BL/6 mice bearing HPV-16 transformed tumors displayed approximately 3 fold higher numbers of CD4⁺/CD25⁺/FoxP3⁺ T regulatory cells in spleen compared to naïve mice or mice whose tumors completely regressed (FIG. 10). The level of T-regs can be reduced in spleen (Panel A and Panel B) or in the tumor (Panel C) by intraperitoneal treatment with cyclophosphamide (100 mg/kg) providing rational for combining chemotherapy with immunotherapy for the treatment of late stage tumors.

### Example 10

### Adjunctive Therapy Significantly Improved Anti-Tumor Efficacy

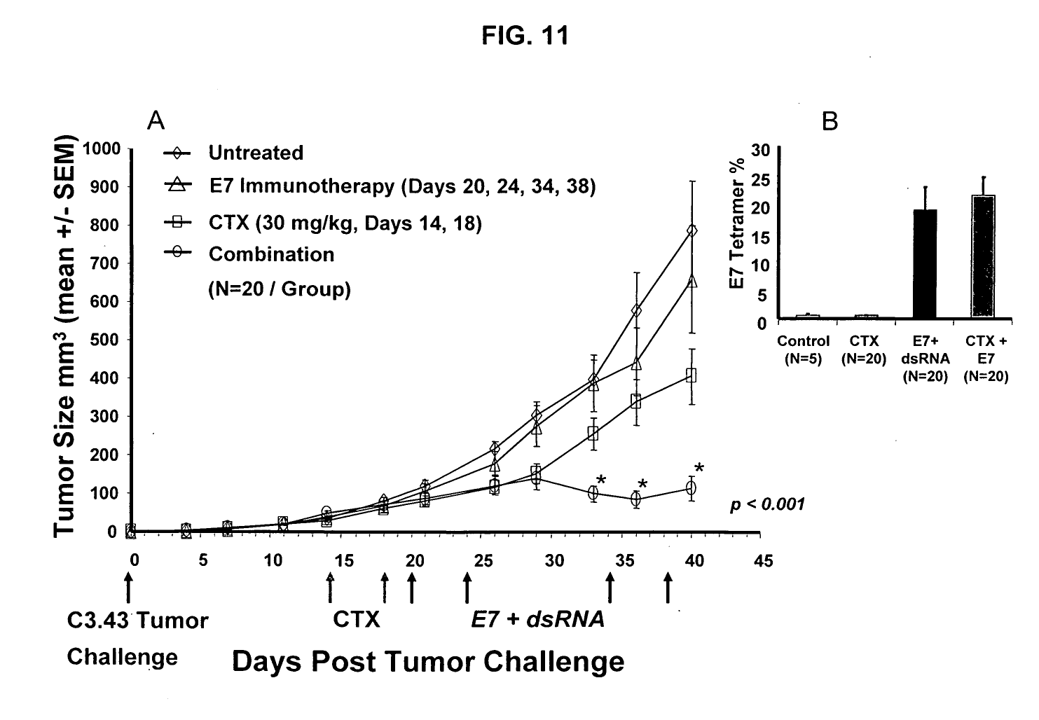

To test efficacy of adjunctive therapy in late stage cancer, C57BL/6 mice were inoculated with 10⁵ HPV-16 transformed tumor cells on Day 0, treated with CTX (30 mg/kg) on Day 14 and 18 (n=20), immunized with E7₄₉₋₅₇ HPV peptide and dsRNA (25 μg/Day) in bilateral inguinal lymph nodes on Day 20, 24, 34, and 38 (n=20), or treated with a combination of CTX and immunotherapy (n=20). Tumor progression (FIG. 11, Panel A) and immune response (FIG. 11, Panel B) was compared to untreated tumor control mice (n=20). The immune response was measured by E7₄₉₋₅₇ Tetramer staining on Day 45 from peripheral blood and the immunized only group displayed HPV specific immune responses in the range of 20% with no observed inhibition of immune response in animals treated with the combination of CTX and immunotherapy which generated a similar response. In addition, the combination of CTX and immunotherapy (Panel A) induced significant tumor regression (p<0.001) compared to immunotherapy and chemotherapy alone which did not significantly induce tumor regression compared to untreated tumor controls.

### Example 11

### Combining Chemotherapy and Immunotherapy Significantly Improved Survival

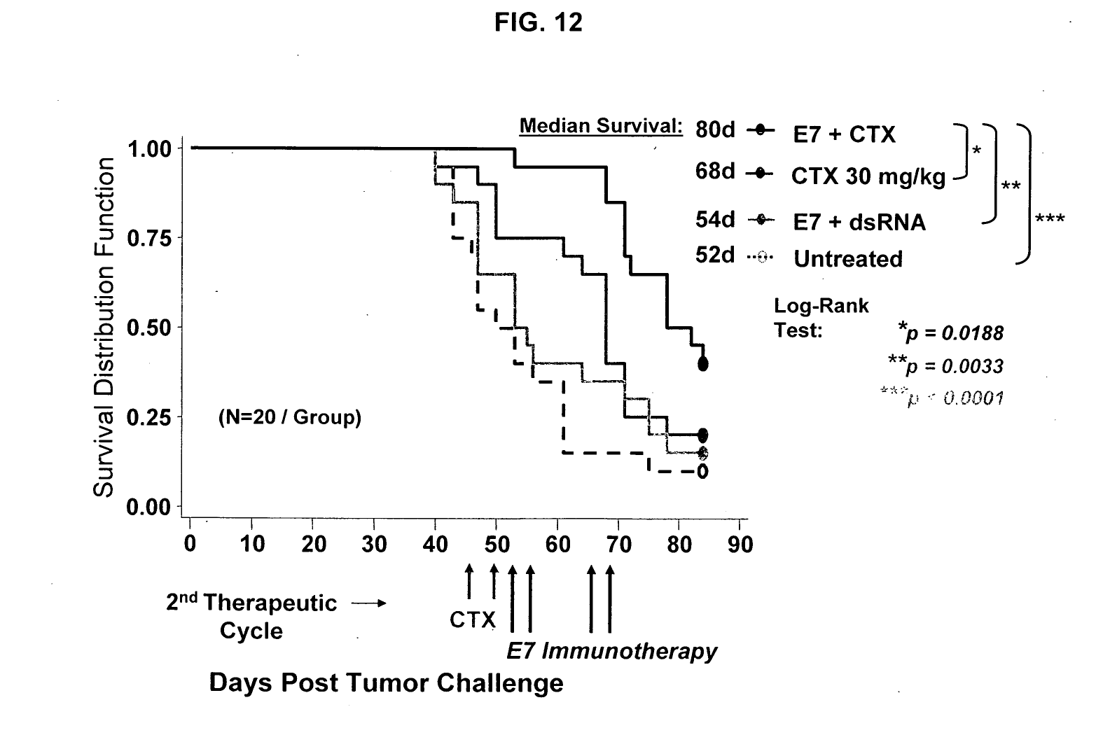

The effect of adjunctive therapy on survival was also evaluated in C57BL/6 mice inoculated with 10⁵ HPV-16 transformed tumor cells as described in Example 10. A second therapeutic cycle was administered in which animals received CTX (30 mg/kg) on Day 46 and 50 (n=20), immunization with E7₄₉₋₅₇ HPV peptide and dsRNA (25 μg/Day) in bilateral inguinal lymph nodes on Day 52, 56, 65, and 69 (n=20), or were treated with a combination of CTX and immunotherapy (n=20). Kaplan-Meier (product-limit) estimates of the survival function were obtained for each of the four conditions (Control, CTX Only, Immunotherapy Only and CTX/Immunotherapy Combined), as shown in FIG. 12. Log-Rank tests were used to compare the four survival curves. The omnibus hypothesis that the four curves are equal was rejected (X²(3)=18.2, p=0.0004). Separate comparisons confirmed that survival in the CTX/Immunotherapy Combined group was significantly longer than survival in the Control group (p<0.0001), the CTX Only group (p=0.0188) and the Immunotherapy Only group (p=0.0033). The median survival time in the CTX/Immunotherapy Combined group was also significantly longer (80 days) compared to the Control group (52 days), the CTX Only group (68 days) and the Immunotherapy Only group (54 days). Therefore, the combination of CTX and HPV immunotherapy significantly improved the disease outcome in later stage cancer compared to either treatment alone.

### Example 12

### Combining Chemotherapy and Subcutaneous Immunotherapy

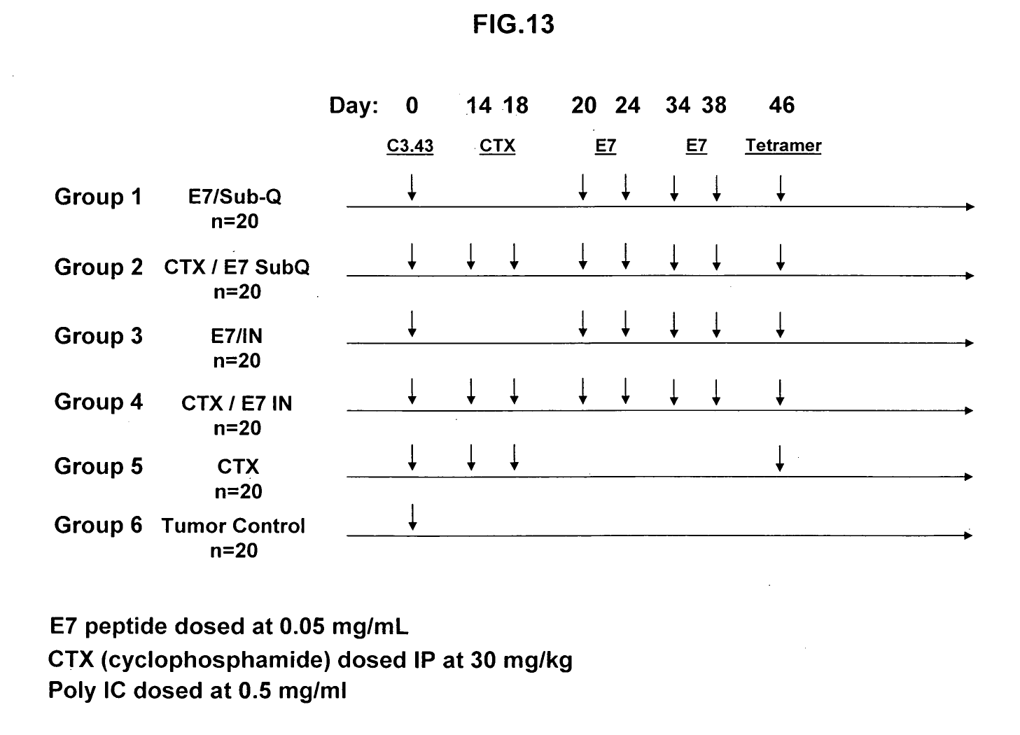

The experiment described in example 10 is repeated with an additional subcutaneous immunotherapy dosing arm and tumor efficacy resulting from subcutaneous versus intra-lymphatic immunotherapy is compared in a setting of combination therapy with CTX. C57BL/6 mice are inoculated with 10⁵ HPV-16 transformed tumor cells on Day 0, treated with CTX (30 mg/kg) on Day 14 and 18 (n=20), immunized subcutaneously or in bilateral inguinal lymph nodes with E7₄₉₋₅₇ HPV peptide and dsRNA (25 μg/Day) on Day 20, 24, 34, and 38 (n=20 per group), or treated with a combination of CTX and subcutaneous or intra-lymphatic immunotherapy (n=20 per group). See FIG. 13 for-adjunctive therapy protocol. Tumor progression and immune response are compared to untreated tumor control mice (n=20). Results emphasize a requirement for CTX followed by intra-lymphatic immunotherapy to elicit significantly superior tumor regression and a survival benefit compared to subcutaneous immunotherapy even in similar combination with CTX.

### Example 13

### Adjuvant Efficacy: Active Immunotherapy Improves Progression Free Survival and Time to Relapse Post Primary Tumor Removal, By Chemotherapy or Surgery

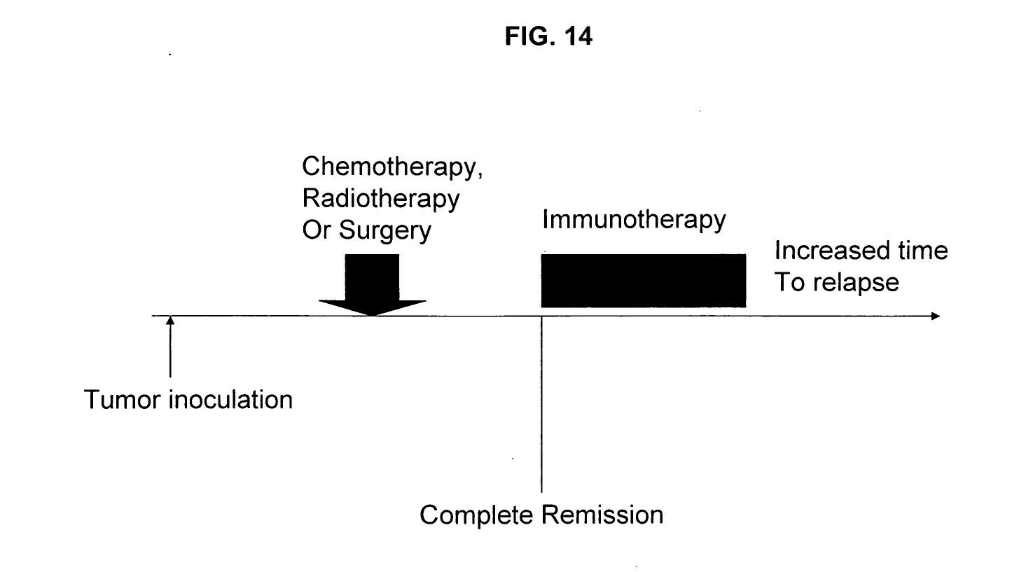

C57BL/6 mice are inoculated subcutaneously with 10⁵ HPV-16 transformed tumor cells on Day 0, and treated with CTX (100 mg/kg) starting on day 14, every other day until they reach complete remission (FIG. 14). A separate cohort is left untreated and tumors are removed at day 20 using surgery or irradiated using conventional radiotherapy. Then all animals are immunized with E7₄₉₋₅₇ HPV peptide and dsRNA (25 μg/Day) in bilateral inguinal lymph nodes on Day 24, 27, 37, and 40 (n=20) and then observed for tumor relapse. Additional control arms are treated with CTX, radiotherapy or surgery but not immunized. Compared to a control cohort (untreated, tumor bearing) that shows 100% tumor formation and progression, all animals treated with CTX, radiotherapy or by surgery attain complete remission (no clinical disease). Nevertheless, without follow up immunotherapy, these animals relapse in a significant number. In contrast, animals that are treated by immunotherapy display a decreased rate of relapse at the site of primary tumor or a remote site, during the same interval and increased median disease free survival. Similar observations are made with a broader range of chemotherapies besides CTX.

### Example 14

### Neoadjuvant Efficacy: Active Immunotherapy Improves the Rate of Response and Shows Clinical Benefit When Applied Prior to Primary Tumor Treatment, by Chemotherapy or Surgery

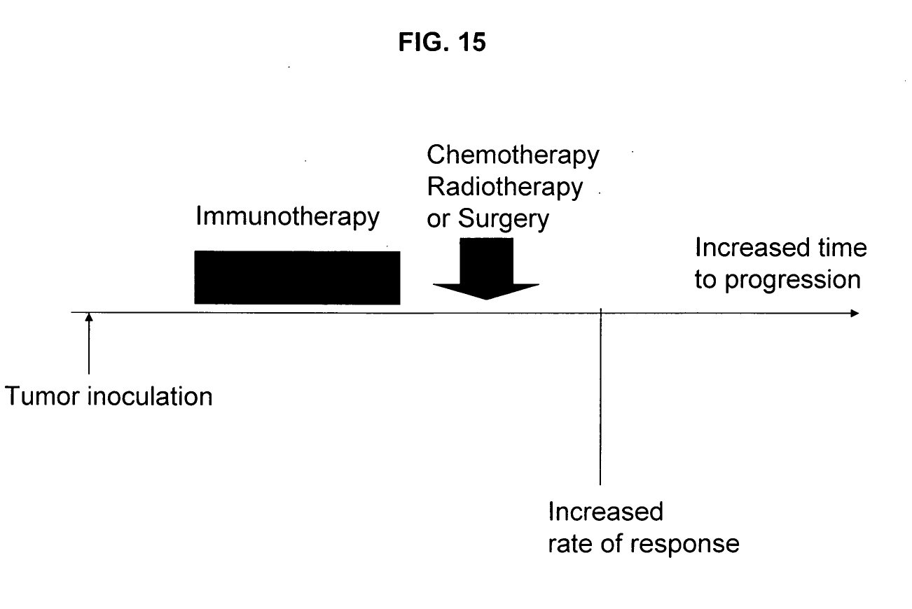

C57BL/6 mice are inoculated subcutaneously with 10⁵ HPV-16 transformed tumor cells on Day 0 then are immunized with E7₄₉₋₅₇ HPV peptide and dsRNA (25 μg/Day) in bilateral inguinal lymph nodes on Day 14, 17, 24, and 27 (n=20). Then mice are treated with CTX (100 mg/kg) starting on day 30 or by radiotherapy, every other day until the animals reach complete remission (FIG. 15). A separate cohort has the tumor removed on day 30 but no treatment with CTX. The animals are then observed for tumor relapse. Compared to a control cohort (tumor bearing and untreated) that shows 100% tumor formation and progression, unimmunized animals treated with CTX, by radiotherapy or by surgery attain partial or complete remission. These animals relapse in a significant number. In contrast, animals that are treated by immunotherapy prior to removing the tumor bulk by surgery, radiotherapy or chemotherapy, display an increased rate of complete and partial remission and a decreased rate of relapse during the same interval, at the same site or a remote site, plus an increased median disease free survival. Similar observations are made with a broader range of chemotherapies besides CTX.

### Example 15

### Consolidation Therapy: Active Immunotherapy Improves Progression Free Survival and Time to Progression Post Chemotherapy

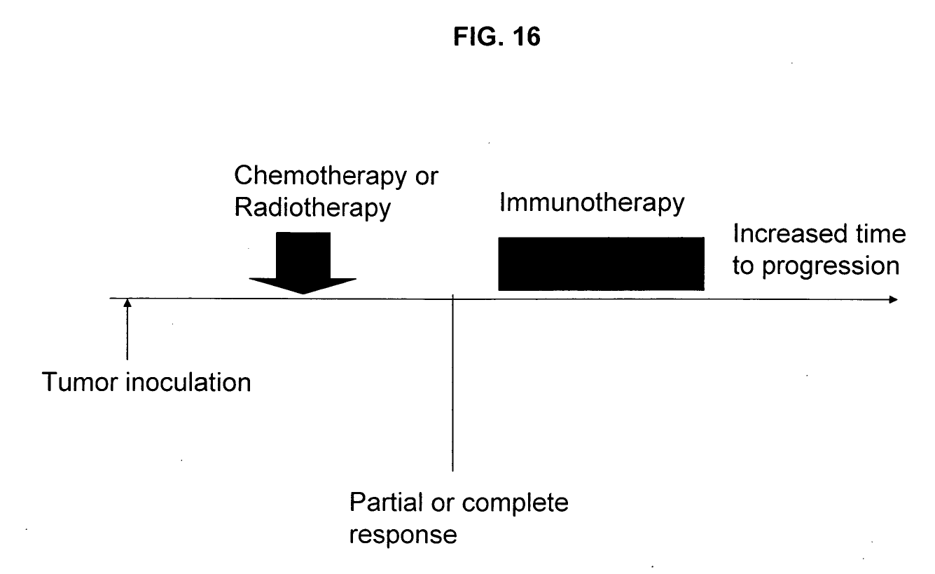

C57BL/6 mice are inoculated subcutaneously with 10⁵ HPV-16 transformed tumor cells on Day 0, and treated with CTX (100 or 30 mg/kg) on days 14 and 16 or treated by radiotherapy (FIG. 16). The animals are rested for 7 days until the number of lymphocytes in the blood reaches normal levels. At that point, all animals show reduced disease or complete remission relative to pre-treatment stage. Then all animals are immunized with E7₄₉₋₅₇ HPV peptide and dsRNA (25 μg/Day) in bilateral inguinal lymph nodes on Day 24, 27, 37, and 40 (n=20) and then observed for tumor reduction and relapse. Additional control arms are treated with CTX or radiotherapy but not immunized. Compared to a control cohort (untreated, tumor bearing) that shows 100% tumor formation and progression, all animals treated with CTX or by radiotherapy attain partial remission or complete remission within 10 days after treatment. Mice immunized show an increased time to progression, progression free survival (if they were in partial remission) and increased time to relapse (if they were in complete remission) compared to animals that are not immunized. Similar observations are made with a broader range of chemotherapies besides CTX.

### Example 16

### Adjunctive Therapy: Active Immunotherapy Improves the Rate of Response When it Accompanies Surgery or Chemotherapy

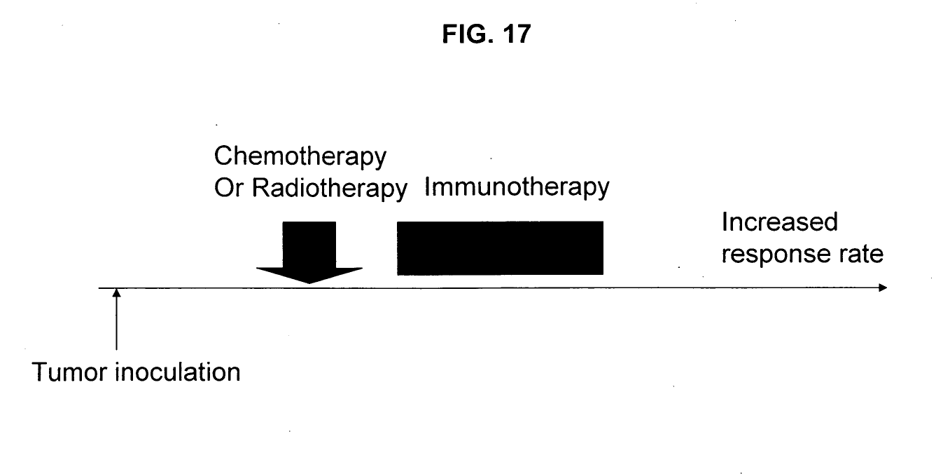

C57BL/6 mice are inoculated subcutaneously with 10⁵ HPV-16 transformed tumor cells on Day 0, and treated with CTX (100 or 30 mg/kg) on days 14 and 16 or treated by radiotherapy. The animals are then immunized with E7₄₉₋₅₇ HPV peptide and dsRNA (25 μg/Day) in bilateral inguinal lymph nodes on Day 18, 21, 28, and 31 (n=20) and then observed for tumor reduction (FIG. 17). Additional control arms are treated with CTX but not immunized. Compared to a control cohort (untreated, tumor bearing) that shows 100% tumor formation and progression, all animals treated with CTX attain partial remission or complete remission within 20 days after CTX treatment. Mice immunized in conjunction with CTX treatment show an increased rate of response (translated into complete or partial response) relative to those treated with CTX or only immunized. Similar observations are made with a broader range of chemotherapies besides CTX.

Any of the methods described in the examples and elsewhere herein can be and are modified to include different compositions, antigens, epitopes, analogues, etc. For example, any other cancer antigen can be used. Also, many epitopes can be interchanged, and the epitope analogues, including those disclosed, described, or incorporated herein by reference can be used. The methods can be used to generate immune responses, including multivalent immune responses against various diseases and illnesses.

Many variations and alternative elements of the invention have been disclosed. Still further variations and alternate elements will be apparent to one of skill in the art. Various embodiments of the invention can specifically include or exclude any of these variation or elements.

What is claimed is:

1. A method of immunization (or cancer treatment) comprising in combination:
contacting a tumor in a patient with a chemotherapeutic agent, wherein the chemotherapeutic agent achieves at least one of promoting tumoral inflammation and interfering with T-regulatory cell function;
inducing a CTL response, wherein the inducing comprises the substeps of:
delivering to the patient a first composition comprising an immunogen, the immunogen comprising or encoding at least a portion of a first antigen, and further comprising an immunopotentiator; and
administering a second composition, comprising an amplifying peptide, directly to a lymphatic system of the patient, wherein the peptide corresponds to an epitope of said first antigen,
wherein the contacting and inducing result in an enhanced effectiveness of treatment beyond the effectiveness of either of the contact step or the inducing step alone.

2. The method of claim 1, wherein the chemotherapeutic agent downregulates or depletes T-regulatory cell activity thereby promoting or enhancing effector T cell activity within a tumor or cancer cell.

3. The method of claim 1, wherein interfering with T-regulatory cell function comprises a reduction in the number of T-regulatory cells.

4. The method of claim 3, wherein the reduction in number of T-regulatory cells is measured using flow cytometry.

5. The method of claim 3, wherein the reduction in number of T-regulatory cells is measured using a marker selected from the group consisting of CD4⁺, CD25⁺, and FoxP3^{HI}.

6. The method of claim 1, wherein interfering with T-regulatory cell function comprises impairing the activity of T-regulatory cells.

7. The method of claim 6, wherein the activity of T-regulatory cells is measured by isolating T-regulatory cells from the patient, incubating the isolated cells with effector cells in a standard assay of effector cell function selected from the group consisting of: a CTL assay, an elispot assay, and a proliferation assay.

8. The method of claim 1, wherein the chemotherapeutic agent is selected from the group consisting of cyclophosphamide, gemcitabine, fludarabine and doxorubicin.

9. The method of claim 8, wherein the chemotherapeutic agent is cyclophosphamide.

10. The method of claim 1 wherein the contacting step is performed upon observation of rising T-regulatory cell function, or induction of abnormal cell proliferation, or tumor growth.

11. The method of claim 1, wherein the contacting and inducing steps are repeated in two or more cycles.

12. The method of claim 11, wherein the contacting and inducing steps are repeated until a reduction in T-regulatory cell activity or a regression of abnormal cell proliferation or tumor growth is achieved.

13. The method of claim 1, wherein contacting step precedes the inducing step.

14. The method of claim 1, wherein the contacting step is repeated prior to the inducing step.

15. The method of claim 1, wherein the contacting step is completed about one week prior to the inducing step.

16. The method of claim 1, wherein the contacting step is repeated prior to the administering substep of the inducing step.

17. The method of claim 1, wherein the delivering substep and the administering substep are carried out on different days.

18. The method of claim 1, wherein the delivering substep of the inducing step occurs after the contacting step.

19. The method of claim 1, wherein the delivering substep includes administering one or more peptides corresponding to an epitope of the antigen prior to or after administering a chemotherapeutic agent.

20. The method of claim 1, further comprising administering at least one mode of treatment selected from the group of radiation therapy, gene therapy, biochemotherapy, and surgery.

21. The method of claim 20, wherein the at least one mode of treatment is provided prior to or during the contacting step.

22. The method of claim 21, wherein the at least one mode of treatment is provided prior to administration of the contacting and inducing steps.
